SEQUENCE LISTING (86 sequences)

SEQ ID NO: 1 - DNA, 65 bp, Artificial Sequence
  Description of Artificial Sequence Synthetic oligonucleotide
nnnnnnnnnnnnnggrccgamaaagvcctgacttctatactaagbctwcgyyccnnnnnnnnnnn

SEQ ID NO: 2 - DNA, 66 bp, Artificial Sequence
  Description of Artificial Sequence Synthetic oligonucleotide
gggaggacgatgcggaccgaaaaagacctgacttctatactaagtctacgttcccagacgactccc

SEQ ID NO: 3 - DNA, 60 bp, Artificial Sequence
  Description of Artificial Sequence Synthetic oligonucleotide
gggacgatgcggaccgaaaaagacctgacttctatactaagtctacgttcccagacgccc

SEQ ID NO: 4 - DNA, 55 bp, Artificial Sequence
  Description of Artificial Sequence Synthetic oligonucleotide
gggatgcggaccgaaaaagacctgacttctatactaagtctacgttcccagaccc

SEQ ID NO: 5 - DNA, 61 bp, Artificial Sequence
  Description of Artificial Sequence Synthetic oligonucleotide
gggacgatgcggaccgaaaaagacctgacttctatactaagtctacgttcccagacgaccc

SEQ ID NO: 6 - DNA, 49 bp, Artificial Sequence
  Description of Artificial Sequence Synthetic oligonucleotide
gggcggaccgaaaaagacctgacttctatactaagtctacgttcccacc

SEQ ID NO: 7 - DNA, 51 bp, Artificial Sequence
  Description of Artificial Sequence Synthetic oligonucleotide
gggcggaccgaaaaagacctgacttctatactaagtctacgttcccagccc

SEQ ID NO: 8 - DNA, 43 bp, Artificial Sequence
  Description of Artificial Sequence Synthetic oligonucleotide
gggaccgaaaaagacctgacttctatactaagtctacgttccc

SEQ ID NO: 9 - DNA, 43 bp, Artificial Sequence
  Description of Artificial Sequence Synthetic oligonucleotide
gggaccgaaaaaggcctgacttctatactaagcctacgttccc

SEQ ID NO: 10 - DNA, 43 bp, Artificial Sequence
  Description of Artificial Sequence Synthetic oligonucleotide
gggaccgaaaaagccctgacttctatactaaggctacgttccc

SEQ ID NO: 11 - DNA, 43 bp, Artificial Sequence
  Description of Artificial Sequence Synthetic oligonucleotide
gggaccgaaaaagacctgacttctatactaagtcttcgttccc

SEQ ID NO: 12 - DNA, 43 bp, Artificial Sequence; identical to SEQ ID NO: 8
  Description of Artificial Sequence Synthetic oligonucleotide

SEQ ID NO: 13 - DNA, 41 bp, Artificial Sequence
  Description of Artificial Sequence Synthetic oligonucleotide
gggccgaaaaagacctgacttctatactaagtctacgtccc

SEQ ID NO: 14 - DNA, 13 bp, Artificial Sequence
  Description of Artificial Sequence Synthetic oligonucleotide
gggaggacgatgc

SEQ ID NO: 15 - DNA, 11 bp, Artificial Sequence
  Description of Artificial Sequence Synthetic oligonucleotide
agacgactccc

SEQ ID NO: 16 - DNA, 10 bp, Artificial Sequence
  Description of Artificial Sequence Synthetic oligonucleotide
gggacgatgc

SEQ ID NO: 17 - DNA, 10 bp, Artificial Sequence
  Description of Artificial Sequence Synthetic oligonucleotide
gggacgatgc

SEQ ID NO: 18 - DNA, 10 bp, Artificial Sequence
  Description of Artificial Sequence Synthetic oligonucleotide
cagacgaccc

SEQ ID NO: 19 - DNA, 33 bp, Artificial Sequence
  Description of Artificial Sequence Synthetic primer
taatacgactcactatagggaggacgatgcgga

SEQ ID NO: 20 - DNA, 16 bp, Artificial Sequence
  Description of Artificial Sequence Synthetic primer
gggagtcgtctgggaa

SEQ ID NO: 21 - DNA, 33 bp, Artificial Sequence
  Description of Artificial Sequence Synthetic primer
taatacgactcactatagggacgatgcggaccg

SEQ ID NO: 22 - DNA, 16 bp, Artificial Sequence
  Description of Artificial Sequence Synthetic primer
gggcgtctgggaacgt

SEQ ID NO: 23 - DNA, 33 bp, Artificial Sequence
  Description of Artificial Sequence Synthetic primer
taatacgactcactatagggatgcggaccgaaa

SEQ ID NO: 24 - DNA, 16 bp, Artificial Sequence
  Description of Artificial Sequence Synthetic primer
gggtctgggaacgtag

SEQ ID NO: 25 - DNA, 16 bp, Artificial Sequence
  Description of Artificial Sequence Synthetic primer
gggtcgtctgggaacg

SEQ ID NO: 26 - DNA, 33 bp, Artificial Sequence
  Description of Artificial Sequence Synthetic primer
taatacgactcactatagggcggaccgaaaaag

SEQ ID NO: 27 - DNA, 16 bp, Artificial Sequence
  Description of Artificial Sequence Synthetic primer
ggtgggaacgtagact

SEQ ID NO: 28 - DNA, 16 bp, Artificial Sequence
  Description of Artificial Sequence Synthetic primer
gggctgggaacgtaga

SEQ ID NO: 29 - DNA, 33 bp, Artificial Sequence
  Description of Artificial Sequence Synthetic primer
taatacgactcactatagggaccgaaaaagacc

SEQ ID NO: 30 - DNA, 16 bp, Artificial Sequence
  Description of Artificial Sequence Synthetic primer
gggaacgtagacttag

SEQ ID NO: 31 - DNA, 60 bp, Artificial Sequence
  Description of Artificial Sequence Synthetic oligonucleotide
taatacgactcactatagggaccgaaaaagacctgacttctatactaagtctacgttccc

SEQ ID NO: 32 - DNA, 60 bp, Artificial Sequence
  Description of Artificial Sequence Synthetic oligonucleotide
gggaacgtagacttagtatagaagtcaggtctttttcggtccctatagtgagtcgtatta

SEQ ID NO: 33 - DNA, 54 bp, Artificial Sequence
  Description of Artificial Sequence Synthetic oligonucleotide
taatacgactcactataggggaaaaagacctgacttctatactaagtctacccc

SEQ ID NO: 34 - DNA, 54 bp, Artificial Sequence
  Description of Artificial Sequence Synthetic oligonucleotide
ggggtagacttagtatagaagtcaggtctttttcccctatagtgagtcgtatta

SEQ ID NO: 35 - DNA, 41 bp, Artificial Sequence
  Description of Artificial Sequence Synthetic oligonucleotide
taatacgactcactatagggcctgacttctatactaagccc

SEQ ID NO: 36 - DNA, 41 bp, Artificial Sequence
  Description of Artificial Sequence Synthetic oligonucleotide
gggcttagtatagaagtcaggccctatagtgagtcgtatta

SEQ ID NO: 37 - DNA, 47 bp, Artificial Sequence
  Description of Artificial Sequence Synthetic oligonucleotide
taatacgactcactatagggaccgaaaaagacctagtctacgttccc

SEQ ID NO: 38 - DNA, 47 bp, Artificial Sequence
  Description of Artificial Sequence Synthetic oligonucleotide
gggaacgtagactaggtctttttcggtccctatagtgagtcgtatta

SEQ ID NO: 39 - DNA, 38 bp, Artificial Sequence
  Description of Artificial Sequence Synthetic oligonucleotide
taatacgactcactatagggaccgaaaaatacgttccc

SEQ ID NO: 40 - DNA, 38 bp, Artificial Sequence
  Description of Artificial Sequence Synthetic oligonucleotide
gggaacgtatttttcggtccctatagtgagtcgtatta

SEQ ID NO: 41 - DNA, 58 bp, Artificial Sequence
  Description of Artificial Sequence Synthetic oligonucleotide
taatacgactcactatagggccgaaaaagacctgacttctatactaagtctacgtccc

SEQ ID NO: 42 - DNA, 58 bp, Artificial Sequence
  Description of Artificial Sequence Synthetic oligonucleotide
gggacgtagacttagtatagaagtcaggtctttttcggccctatagtgagtcgtatta

SEQ ID NO: 43 - DNA, 60 bp, Artificial Sequence
  Description of Artificial Sequence Synthetic oligonucleotide
taatacgactcactatagggaccgaaaaaggcctgacttctatactaagcctacgttccc

SEQ ID NO: 44 - DNA, 60 bp, Artificial Sequence
  Description of Artificial Sequence Synthetic oligonucleotide
gggaacgtaggcttagtatagaagtcaggcctttttcggtccctatagtgagtcgtatta

SEQ ID NO: 45 - DNA, 60 bp, Artificial Sequence
  Description of Artificial Sequence Synthetic oligonucleotide
taatacgactcactatagggaccgaaaaagccctgacttctatactaaggctacgttccc

SEQ ID NO: 46 - DNA, 60 bp, Artificial Sequence
  Description of Artificial Sequence Synthetic oligonucleotide
gggaacgtagccttagtatagaagtcagggctttttcggtccctatagtgagtcgtatta

SEQ ID NO: 47 - DNA, 60 bp, Artificial Sequence
  Description of Artificial Sequence Synthetic oligonucleotide
taatacgactcactatagggaccgaaaaagacctgacttctatactaagtctacggtccc

SEQ ID NO: 48 - DNA, 60 bp, Artificial Sequence
  Description of Artificial Sequence Synthetic oligonucleotide
gggaccgtagacttagtatagaagtcaggtctttttcggtccctatagtgagtcgtatta

SEQ ID NO: 49 - DNA, 60 bp, Artificial Sequence
  Description of Artificial Sequence Synthetic oligonucleotide
taatacgactcactatagggaccgaaaaagacctgacttctatactaagtcttcgttccc

SEQ ID NO: 50 - DNA, 60 bp, Artificial Sequence
  Description of Artificial Sequence Synthetic oligonucleotide
gggaacgaagacttagtatagaagtcaggtctttttcggtccctatagtgagtcgtatta

SEQ ID NO: 51 - DNA, 60 bp, Artificial Sequence
  Description of Artificial Sequence Synthetic oligonucleotide
taatacgactcactatagggaccgaaaaagacctgacttctatactaggtctacgttccc

SEQ ID NO: 52 - DNA, 60 bp, Artificial Sequence
  Description of Artificial Sequence Synthetic oligonucleotide
gggaacgtagacctagtatagaagtcaggtctttttcggtccctatagtgagtcgtatta

SEQ ID NO: 53 - DNA, 60 bp, Artificial Sequence
  Description of Artificial Sequence Synthetic oligonucleotide
taatacgactcactatagggaccgaaaaagacctggcttctatactaagtctacgttccc

SEQ ID NO: 54 - DNA, 60 bp, Artificial Sequence
  Description of Artificial Sequence Synthetic oligonucleotide
gggaacgtagacttagtatagaagccaggtctttttcggtccctatagtgagtcgtatta

SEQ ID NO: 55 - DNA, 60 bp, Artificial Sequence
  Description of Artificial Sequence Synthetic oligonucleotide
taatacgactcactatagggaccgaaaaagacctgacttctatactaagtctacgatccc

SEQ ID NO: 56 - DNA, 60 bp, Artificial Sequence
  Description of Artificial Sequence Synthetic oligonucleotide
gggatcgtagacttagtatagaagtcaggtctttttcggtccctatagtgagtcgtatta

SEQ ID NO: 57 - DNA, 60 bp, Artificial Sequence
  Description of Artificial Sequence Synthetic oligonucleotide
taatacgactcactatagggaccgaaaaagacctgacttctatactaagtctacgctccc

SEQ ID NO: 58 - DNA, 60 bp, Artificial Sequence
  Description of Artificial Sequence Synthetic oligonucleotide
gggagcgtagacttagtatagaagtcaggtctttttcggtccctatagtgagtcgtatta

SEQ ID NO: 59 - RNA, 70 nt, Artificial Sequence
  Description of Artificial Sequence Synthetic oligonucleotide
gggaggacgaugcggaccgaaaaagaccugacuucuauacuaagucuacguucccagacgacucgcccga

SEQ ID NO: 60 - DNA, 49 bp, Artificial Sequence
  Description of Artificial Sequence Synthetic primer
gggggaattctaatacgactcactatagggagagaggaagagggatggg

SEQ ID NO: 61 - DNA, 34 bp, Artificial Sequence
  Description of Artificial Sequence Synthetic primer
ggggggatccagtactatcgacctctgggttatg

SEQ ID NO: 62 - DNA, 32 bp, Artificial Sequence
  Description of Artificial Sequence Synthetic primer
taatacgactcactatagggaggacgatgcgg

SEQ ID NO: 63 - DNA, 18 bp, Artificial Sequence
  Description of Artificial Sequence Synthetic primer
aggagtgacgtaaacatg

SEQ ID NO: 64 - DNA, 32 bp, Artificial Sequence
  Description of Artificial Sequence Synthetic primer
taatacgactcactataggggcatgcctagct

SEQ ID NO: 65 - DNA, 18 bp, Artificial Sequence
  Description of Artificial Sequence Synthetic primer
ccgcgcataagccatggg

SEQ ID NO: 66 - RNA, 66 nt, Artificial Sequence
  Description of Artificial Sequence Synthetic oligonucleotide
gggaggacgaugcggaccgaaaaagaccugacuucuauacuaagucuacguucccagacgacuccc

SEQ ID NO: 67 - RNA, 60 nt, Artificial Sequence
  Description of Artificial Sequence Synthetic oligonucleotide
gggacgaugcggaccgaaaaagaccugacuucuauacuaagucuacguucccagacgccc

SEQ ID NO: 68 - RNA, 55 nt, Artificial Sequence
  Description of Artificial Sequence Synthetic oligonucleotide
gggaugcggaccgaaaaagaccugacuucuauacuaagucuacguucccagaccc

SEQ ID NO: 69 - RNA, 61 nt, Artificial Sequence
  Description of Artificial Sequence Synthetic oligonucleotide
gggacgaugcggaccgaaaaagaccugacuucuauacuaagucuacguucccagacgaccc

SEQ ID NO: 70 - RNA, 49 nt, Artificial Sequence
  Description of Artificial Sequence Synthetic oligonucleotide
gggcggaccgaaaaagaccugacuucuauacuaagucuacguucccacc

SEQ ID NO: 71 - RNA, 51 nt, Artificial Sequence
  Description of Artificial Sequence Synthetic oligonucleotide
gggcggaccgaaaaagaccugacuucuauacuaagucuacguucccagccc

SEQ ID NO: 72 - RNA, 43 nt, Artificial Sequence
  Description of Artificial Sequence Synthetic oligonucleotide
gggaccgaaaaagaccugacuucuauacuaagucuacguuccc

SEQ ID NO: 73 - RNA, 37 nt, Artificial Sequence
  Description of Artificial Sequence Synthetic oligonucleotide
ggggaaaaagaccugacuucuauacuaagucuacccc

SEQ ID NO: 74 - RNA, 24 nt, Artificial Sequence
  Description of Artificial Sequence Synthetic oligonucleotide
gggccugacuucuauacuaagccc

SEQ ID NO: 75 - RNA, 30 nt, Artificial Sequence
  Description of Artificial Sequence Synthetic oligonucleotide
gggaccgaaaaagaccuagucuacguuccc

SEQ ID NO: 76 - RNA, 21 nt, Artificial Sequence
  Description of Artificial Sequence Synthetic oligonucleotide
gggaccgaaaaauacguuccc

SEQ ID NO: 77 - RNA, 43 nt, Artificial Sequence
  Description of Artificial Sequence Synthetic oligonucleotide
gggaccgaaaaaggccugacuucuauacuaagccuacguuccc

SEQ ID NO: 78 - RNA, 43 nt, Artificial Sequence
  Description of Artificial Sequence Synthetic oligonucleotide
gggaccgaaaaagcccugacuucuauacuaaggcuacguuccc

SEQ ID NO: 79 - RNA, 43 nt, Artificial Sequence
  Description of Artificial Sequence Synthetic oligonucleotide
gggaccgaaaaagaccugacuucuauacuaagucuacgguccc

SEQ ID NO: 80 - RNA, 43 nt, Artificial Sequence
  Description of Artificial Sequence Synthetic oligonucleotide
gggaccgaaaaagaccugacuucuauacuaagucuucguuccc

SEQ ID NO: 81 - RNA, 43 nt, Artificial Sequence
  Description of Artificial Sequence Synthetic oligonucleotide
gggaccgaaaaagaccugacuucuauacuaggucuacguuccc

SEQ ID NO: 82 - RNA, 43 nt, Artificial Sequence
  Description of Artificial Sequence Synthetic oligonucleotide
gggaccgaaaaagaccuggcuucuauaccaagucuacguuccc

SEQ ID NO: 83 - RNA, 43 nt, Artificial Sequence
  Description of Artificial Sequence Synthetic oligonucleotide
gggaccgaaaaagaccugacuucuauacuaagucuacgauccc

SEQ ID NO: 84 - RNA, 43 nt, Artificial Sequence
  Description of Artificial Sequence Synthetic oligonucleotide
gggaccgaaaaagaccugacuucuauacuaagucuacgcuccc

SEQ ID NO: 85 - RNA, 43 nt, Artificial Sequence
  Description of Artificial Sequence Synthetic oligonucleotide
gggaccgacaaagaccugacuucuauacuaagucuacgcuccc

SEQ ID NO: 86 - RNA, 41 nt, Artificial Sequence
  Description of Artificial Sequence Synthetic oligonucleotide
gggccgaaaaagaccugacuucuauacuaagucuacguccc

(19) United States
(12) Patent Application Publication
(10) Pub. No.: US 2014/0148503 A1
(43) Pub. Date: May 29, 2014

(54) NUCLEIC ACID APTAMERS

(71) Applicants: Paloma H. Giangrande, Iowa City, IA (US); William M. Rockey, Iowa City, IA (US)

(72) Inventors: Paloma H. Giangrande, Iowa City, IA (US); William M. Rockey, Iowa City, IA (US)

(73) Assignee: University of Iowa Research Foundation, Iowa City, IA (US)

(21) Appl. No.: 14/233,867

(22) PCT Filed: Jul. 18, 2012
(86) PCT No.: PCT/US2012/047196

§ 371 Date: Jan. 20, 2014

Related U.S. Application Data
(60) Provisional application No. 61/509,938, filed on Jul. 20, 2011.

(51) Int. Cl. C12N 15/115; G01N 33/574

(52) U.S. Cl. CPC C12N 15/115; G01N 33/574
USPC 514/44 R; 536/23.1; 536/24.5; 435/320.1; 435/375; 436/501

(57) ABSTRACT

The present invention relates to optimized aptamers and methods of using these aptamers.

### RELATED APPLICATION

This application claims priority under 35 U.S.C. 119(e) to provisional application U.S. Ser. No. 61/509,938, filed Jul. 20, 2011, which application is incorporated hereby by reference.

### STATEMENT REGARDING FEDERALLY SPONSORED RESEARCH

The invention was made with Government support under National Institutes of Health Grant Nos. 1RO1CA138503-01, 1R21DE019953-01. The government has certain rights in the invention.

### BACKGROUND OF THE INVENTION

Worldwide, cancer affects approximately 10 million people each year. Approximately 22 million people are living with cancer and almost 7 million people die worldwide from cancer each year. The most common cancers include cancers of the lung, breast, colon/rectum, stomach, liver, prostate, cervix, esophagus, and bladder. The elderly tend to be the highest population for new incidence, as more than 75% of all new cancer cases are diagnosed in people over the age of 60. With the aging population, incidence is expected to increase each year. Prostate cancer is the most common cancer in men and the second leading cause of cancer death in men, behind lung cancer. Approximately 80% of prostate cancers are diagnosed in men over 65 years of age, and, due to the lack of symptoms, 75% of first-time patients over 65 are diagnosed with Stage C or D, the two most advanced stages of prostate cancer. Worldwide, more than 680,000 men are diagnosed annually. Prostate cancer characteristically spreads to the bone.

RNA interference (RNAi) is a cellular mechanism by which 21-23 nt RNA duplexes trigger the degradation of cognate mRNAs. Researchers have been pursuing potential therapeutic applications of RNAi once it was demonstrated that exogenous, short interfering RNAs (siRNAs) can silence gene expression via this pathway in mammalian cells. RNAi is attractive for therapeutics because of its stringent target gene specificity, the relatively low immunogenicity of siRNAs, and the simplicity of design and testing of siRNAs.

Double-stranded RNA (dsRNA) can induce sequence-specific posttranscriptional gene silencing in many organisms by a process known as RNA interference (RNAi). However, in mammalian cells, dsRNA that is 30 base pairs or longer can induce sequence-nonspecific responses that trigger a shut-down of protein synthesis. RNA fragments are the sequence-specific mediators of RNAi. Interference of gene expression by these RNA interference (RNAi) molecules is now recognized as a naturally occurring strategy for silencing genes in the cells of many organisms.

One technical hurdle for RNAi-based clinical applications that still remains is the delivery of siRNAs across the plasma membrane of cells in vivo. A number of solutions for this problem have been described. However, most of the approaches described to date have the disadvantage of delivering siRNAs to cells non-specifically, without regard to the cell type.

For in vivo use, the therapeutic siRNA reagents need to target particular cell types (e.g., cancer cells), thereby limiting side-effects that result from non-specific delivery as well as reducing the quantity of siRNA necessary for treatment.

### SUMMARY OF THE INVENTION

Accordingly, in certain embodiments, the present invention provides a nucleic acid aptamer molecule of 41 to 66 nucleotides in length comprising the nucleic acid sequence 5′-N₁GGRCCGAMAAAGVCCTGACTTCTATACTAAGBCTWCGYYCCN₂-3′ (SEQ ID NO:1), where N₁ and N₂ can be present or absent, wherein when present N₁ is GGGAGGACGATGC and N₂ is AGACGACTCCC, N₁ is GGGACGATGC and N₂ is CAGACGCCC, N₁ is GGGATGC and N₂ is CAGACCC, N₁ is GGGACGATGC and N₂ is CAGACGACCC, N₁ is GGGC and N₂ is CACC, N₁ is GGGC and N₂ is CAGCCC, or N₁ is G and N₂ is C, wherein R is a G or A nucleotide, wherein M is a A or C nucleotide, wherein V is a A, G or C nucleotide, wherein B is a T, C or G nucleotide, wherein W is a A or T nucleotide, wherein Y is T or C nucleotide, or its complement, or an RNA equivalent of the molecule or its complement.

In certain embodiments, the nucleic acid aptamer molecule is capable of binding to PSMA with high affinity and specificity. As used herein, the term “high affinity” means that the aptamer binds to the target PSMA in a low nM to pM range. As used herein, the term “specificity” means that the aptamer binds to the target PSMA in a low nM to pM range. In certain embodiments, the nucleic acid aptamer molecule is capable of inhibiting PSMA enzymatic activity. The present invention provides a method of inhibiting PSMA enzymatic activity in a mammal in need thereof; e.g., by introducing the aptamer in an amount sufficient to inhibit PSMA enzymatic activity. The PSMA enzyme can be inhibited by at least 1-100%, such as by 10%, 20%, 30%, 40%, 50%, 60%, 70%, 80%, 90%, 95%, or 99%.

In certain embodiments, the present invention provides a nucleic acid aptamer molecule consisting of A9a (SEQ ID NO:2), A9b (SEQ ID NO:3), A9c (SEQ ID NO:4), A9d (SEQ ID NO:5), A9e (SEQ ID NO:6), A9f (SEQ ID NO:7), A9g (SEQ ID NO:8), A9g.1 (SEQ ID NO:9), A9g.2 (SEQ ID NO:10), A9g.4 (SEQ ID NO:11), A9g.9/A9C (SEQ ID NO:12), or A9L (SEQ ID NO:13), or its complement, or an RNA equivalent of the molecule or its complement.

In certain embodiments, the present invention consists of A9L (SEQ ID NO:13), or its complement, or an RNA equivalent of the molecule or its complement. In certain embodiments, the present invention consists of A9g (SEQ ID NO:8), or its complement, or an RNA equivalent of the molecule or its complement.

In certain embodiments, the nucleic acid of the present invention is RNA. In certain embodiments, the nucleic acid of the present invention is DNA. In certain embodiments, the nucleic acid molecule includes a modified nucleotide. In certain embodiments, the present invention provides a conjugate comprising the nucleic acid molecule described above linked to a therapeutic or diagnostic molecule. In certain embodiments, “linked” includes directly linking (covalently or non-covalently binding) the nucleic acid molecule of the invention (e.g., an aptamer) to a therapeutic or diagnostic molecule. In certain embodiments, “linked” includes linking the nucleic acid molecule of the invention (e.g., an aptamer) to a therapeutic or diagnostic molecule using a linker, e.g., a nucleotide linker, e.g., the nucleotide sequence “AA” or “TT” or “UU”. In certain embodiments, the therapeutic molecule is a siRNA molecule. In certain embodiments, the conjugate further comprises a PEG molecule. In certain embodiments, the PEG molecule has an average molecular weight of about 10 to 100 kDa in size. In certain embodiments, the PEG molecule has an average molecular weight of about 10 to 40 kDa in size. In certain embodiments, the PEG molecule is PEG-20.

The present invention further provides a nucleic acid coding molecule encoding a nucleic acid aptamer molecule as described above. The present invention further provides an expression cassette comprising the nucleic acid coding molecule described above. In certain embodiments, the expression cassette further includes a promoter, such as a regulatable promoter or a constitutive promoter. Examples of suitable promoters include a CMV, RSV, pol II or pol III promoter. The expression cassette may further contain a polyadenylation signal (such as a synthetic minimal polyadenylation signal) and/or a marker gene. Examples of marker genes include visual markers such as GFP, or functional markers, such as antibiotic resistance genes.

In certain embodiments, the expression cassette is contained in a vector, such as a viral vector or a plasmid vector. Certain embodiments of the invention provide a vector, e.g., a viral vector, including at least one (e.g., 1 or 2) expression cassette of the invention. Examples of appropriate vectors include adenoviral, lentiviral, adeno-associated viral (AAV), poliovirus, HSV, or murine Maloney-based viral vectors. In one embodiment, the vector is an adenoviral vector. In certain embodiments, a vector may contain two expression cassettes.

Certain embodiments of the invention provide an isolated or non-human cell including the PMSA receptor and a molecule or conjugate of the invention.

Certain embodiments of the invention provide methods for delivering a therapeutic or diagnostic molecule to a cell having a PMSA receptor, including contacting the cell with a conjugate of the invention.

The present invention further provides a pharmaceutical composition comprising a molecule or conjugate as described above and a pharmaceutically acceptable carrier.

Certain embodiments of the invention provide a method for treating a patient having cancer including administering a molecule, duplex or conjugate of the invention to the patient.

Certain embodiments of the invention provide a method for determining whether a patient has cancer (i.e., diagnosing a patient) including administering a conjugate of the invention to the patient and determining whether the patient has cancer. For example, because certain conjugates of the invention are targeted to the PMSA receptor and include a diagnostic molecule, detection of a relatively higher level of the conjugate can be used to diagnose a patient as having prostate cancer.

The present invention further provides a use of a molecule or conjugate as described above for treating cancer. In certain embodiments, the cancer is a solid sarcoma or carcinoma. In certain embodiments, the cancer is prostate cancer.

The present invention further provides a molecule or conjugate as described above for use in therapy.

The present invention further provides a molecule or conjugate as described above for use in the prophylactic or therapeutic treatment of cancer.

In certain embodiments, the therapeutic molecule is an RNAi molecule, such as a siRNA molecule, e.g., a siRNA molecule targeted to polo-like kinase 1 (PLK1). While certain exemplary siRNA sequences have been utilized herein, the invention is also directed to the use of other siRNA sequences, for example, siRNA sequences that target genes involved in cancer. In certain embodiments, the therapeutic molecule is a microRNA (miRNA).

The present invention relates to a specific delivery of siRNAs and one that, at least in one embodiment, only uses properties of RNA. The delivery method of the instant invention exploits the structural potential of nucleic acids (e.g., RNA) to target siRNAs to a particular cell-surface receptor and thus to a specific cell type. In one embodiment, the invention provides a method and compositions to specifically deliver nucleic acids that comprise both a targeting moiety (e.g., an aptamer) and an RNA-silencing moiety (e.g., an siRNA) that is recognized and processed by Dicer in a manner similar to the processing of microRNAs. Aptamers and siRNAs have low immunogenicity. They can easily be synthesized in large quantities at a relatively low cost and are amendable to a variety of chemical modifications that confer both resistance to degradation and improved pharmacokinetics in vivo. The smaller size of aptamers compared with that of antibodies (<15 kDa versus 150 kDa) facilitates their in vivo delivery by promoting better tissue penetration.

In certain embodiments of the invention, RNAi molecules are employed to inhibit expression of a target gene. By “inhibit expression” is meant to reduce, diminish or suppress expression of a target gene. Expression of a target gene may be inhibited via “gene silencing.” Gene silencing refers to the suppression of gene expression, e.g., transgene, heterologous gene and/or endogenous gene expression, which may be mediated through processes that affect transcription and/or through processes that affect post-transcriptional mechanisms. In some embodiments, gene silencing occurs when an RNAi molecule initiates the degradation of the mRNA transcribed from a gene of interest in a sequence-specific manner via RNA interference, thereby preventing translation of the gene's product.

As used herein the term “encoded by” is used in a broad sense, similar to the term “comprising” in patent terminology. For example, the statement “the first strand of RNA is encoded by SEQ ID NO:1” means that the first strand of RNA sequence corresponds to the RNA sequence transcribed from the DNA sequence indicated in SEQ ID NO:1, but may also contain additional nucleotides at either the 3′ end or at the 5′ end of the RNA molecule.

The reference to siRNAs herein is meant to include short hairpin RNAs (shRNAs) and other small RNAs that can or are capable of modulating the expression of a target gene, for example via RNA interference. Such small RNAs include without limitation, shRNAs and miroRNAs (miRNAs).

The two strands of RNA in the siRNA may be completely complementary, or one or the other of the strands may have an “overhang region” (i.e., a portion of the RNA that does not bind with the second strand). Such an overhang region may be from 1 to 10 nucleotides in length.

This invention relates to compounds, compositions, and methods useful for inhibiting a target gene expression using short interfering nucleic acid (siRNA) molecules. This invention also relates to compounds, compositions, and methods useful for modulating the expression and activity of the target gene by RNA interference (RNAi) using small nucleic acid molecules. In particular, the instant invention features small nucleic acid molecules, such as short interfering nucleic acid (siNA), short interfering RNA (siRNA), double-stranded RNA (dsRNA), micro-RNA (miRNA), and short hairpin RNA (shRNA) molecules and methods used to modulate the expression of target genes. A siRNA of the instant invention can be chemically synthesized, expressed from a vector or enzymatically synthesized.

In the present invention, an expression cassette may contain a nucleic acid encoding at least one strand of the RNA duplex described above. Such an expression cassette may further contain a promoter. The expression cassette may be contained in a vector. These cassettes and vectors may be contained in a cell, such as a mammalian cell. A non-human mammal may contain the cassette or vector. The vector may contain two expression cassettes, the first expression cassette containing a nucleic acid encoding the first strand of the RNA duplex, and a second expression cassette containing a nucleic acid encoding the second strand of the RNA duplex.

The present invention further provides a method of substantially silencing a target gene of interest or targeted allele for the gene of interest in order to provide a therapeutic effect. As used herein the term “substantially silencing” or “substantially silenced” refers to decreasing, reducing, or inhibiting the expression of the target gene or target allele by at least about 5%, 10%, 15%, 20%, 25%, 30%, 35%, 40%, 45%, 50%, 55%, 60%, 65%, 70%, 75%, 80%, 85% to 100%. As used herein the term “therapeutic effect” refers to a change in the associated abnormalities of the disease state, including pathological and behavioral deficits; a change in the time to progression of the disease state; a reduction, lessening, or alteration of a symptom of the disease; or an improvement in the quality of life of the person afflicted with the disease. Therapeutic effect can be measured quantitatively by a physician or qualitatively by a patient afflicted with the disease state targeted by the siRNA. In certain embodiments wherein both the mutant and wild type allele are substantially silenced, the term therapeutic effect defines a condition in which silencing of the wild type allele's expression does not have a deleterious or harmful effect on normal functions such that the patient would not have a therapeutic effect.

In one embodiment, the expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is flanked (5′ and 3′) with functional sequences, such as sequences encoding an aptamer and/or siRNA.

In one embodiment, the selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from nonviral genes, such as the murine metallothionein gene, will also find use herein. Such promoter sequences are commercially available from, e.g., Stratagene® (San Diego, Calif.).

In one embodiment, pharmaceutical compositions will comprise sufficient genetic material to produce a therapeutically effective amount of the siRNA of interest, i.e., an amount sufficient to reduce or ameliorate symptoms of the disease state in question or an amount sufficient to confer the desired benefit. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

### BRIEF DESCRIPTION OF DRAWINGS

FIGS. 1A-1D. Functional characterization of various truncations of the A9 PSMA RNA aptamers generated using RNA secondary structural prediction algorithms. (A) RNA aptamers A9, A10, A10-3, and A10-3.2 were incubated with recombinant PSMA protein. Production of [³H]-glutamate from [³H]-NAAG was measured using a NAALADase assay. RNA aptamers A10 scrambled and A10-3.2-scrambled were used as negative controls in this assay. NAALADase activity in the presence of each RNA was normalized to the no RNA sample (−RNA). (B) Secondary structural predictions of truncated A9 aptamers generated using the RNAStructure 4.6 algorithm. Base changes are denoted by an asterisk (*). Base changes were introduced to retain a leading GGG transcription start codon at the 5′ end of the truncated RNA sequences or in order to maintain base complementarity at the 3′ end. (C) Effect of A9 aptamer and truncated derivatives of the A9 aptamer on PSMA enzymatic activity. NAALADase activity was normalized as in part (A) above. (D) A9 and A9g RNA aptamers inhibit PSMA NAALADase enzymatic activity with approximate IC₅₀ values of 10 nM.

FIGS. 2A-2B. Further truncation of the A9 aptamer causes loss of inhibitory activity. (A) Secondary structural predictions of truncated A9 aptamers generated using the RNAStructure 4.6 algorithm. Base changes are denoted by an asterisk (*). Base changes were introduced to retain a leading GGG transcription start codon at the 5′ end of the truncated RNA sequences or in order to maintain base complementarity at the 3′ end. (B) Effect of A9 aptamer and truncated derivatives of the A9 aptamer on PSMA enzymatic activity. NAALADase activity was normalized as in FIG. 1.

FIGS. 3A-3B. Binding of A9 and A9g to human PSMA. (A) A saturation filter binding assay was used to measure binding of A9 and A9g to recombinant human PSMA protein. The calculated _{KD} for A9 was 110 nM and the _{KD} for A9g was 130 nM. The fraction bound was normalized to the Bₘₐₓ (maximal binding capacity) of A9. (B) Measurement of the binding affinity of A9g for recombinant human PSMA protein by surface plasmon resonance (SPR, BIACore). The data were fit to a 1:1 binding with mass transfer model. The _{KD} of A9g calculated from the model was 5 nM with a χ² value of 1.51. The on-rate (kₐ) was 1.15×10⁴ M⁻¹·S⁻¹ and the off-rate (_{kd}) was 5.7×10⁻⁵ s⁻¹.

FIGS. 4A-4D. Characterization of A9g binding to PSMA. (A) Secondary structural predictions of truncated A9 aptamers generated using the RNAStructure 4.6 algorithm. Base changes are denoted by an asterisk (*). Base changes were introduced in an attempt to either retain the predicted secondary structure (A9g.1 and A9g.2) or disrupt various secondary structural elements (A9g.3-A9g.6) of A9g. Two secondary structure predictions were given for the A9g.4 sequence, denoted by A9g.4a and A9g.4b. (B) Effect of A9g aptamer derivatives (A9g.1 through A9g.6) on PSMA NAALADase inhibitor activity. NAALADase activity was measured and normalized as in FIG. 1. (C) Saturation filter binding assay of A9g aptamer and A9g aptamer derivatives (A9g.3-A9g.6). (D) Binding of A9g to recombinant human PSMA, recombinant rat HER2 (rHER2), and BSA using BIACore (left panel). Binding of A9g.6 to recombinant human PSMA, recombinant rat HER2 (rHER2), and BSA using BIACore (right panel).

FIGS. 5A-5C. Truncated A9 PSMA aptamers derived based on RNA tertiary structure and protein/RNA docking predictions. (A) Modeled tertiary structure of A9g docked to a crystal structure of PSMA. The bases A9 and U39 are predicted to form direct interactions with the crystal structure of PSMA. The amine group of A9 is predicted to form a hydrogen bond with a backbone carbonyl of PSMA (close up; middle panel). Right panel; close up of A9g (A9C) variant where the A at position 9 was changed to a C to retain the hydrogen bond. (B) Secondary structural predictions of A9g aptamer and A9g aptamer derivatives generated using the RNAStructure 4.6 algorithm (left panel). Base changes are denoted by an asterisk (*). Secondary structural predictions of A9g were generated to test the importance of the uracil at position 39 and the adenosine at position 9. Effect of A9g and A9g aptamer derivatives (U39A, U39C, U39G, and A9C) on PSMA NAALADase activity (right panel). (C) Secondary structural predictions of A9g aptamer and truncated derivatives A9L (41 mer) and A9h (37 mer) using the RNAStructure 4.6 algorithm (left panel). Base changes are denoted by an asterisk (*). Effect of A9L (41 mer) and A9h (37 mer) aptamers on PSMA NAALADase activity. NAALADase activity was measured and normalized as in FIG. 1 (right panel).

FIGS. 6A-6B. Truncated A9 aptamers bind to and internalize into PSMA expressing cells. (A) Internalization of PSMA RNA aptamers A9, A9g (43 mer), A9L (41 mer) and A9g.6 into prostate cancer cells expressing PSMA. Internalization was measured using quantitative RT-PCR. RNA recovery was normalized to recovery of an internal RNA control. (B) Internalization of PSMA RNA aptamers A10, A9 and derivatives into PSMA expressing prostate cancer cells. A10-3.2 scrambled and A9g.6 aptamers were used as negative controls for internalization in this assay. The fold enrichment in recovery with respect to non-PSMA expressing cells is reported.

FIG. 7. Binding of A9g to PSMA-expressing prostate cancer cells. Varying concentrations of ³²P-end-labeled A9g were incubated with either PSMA-expressing cells (22Rv1 [1.7]) or non-expressing cells (PC3). The incubation was performed at 4° C. to prevent internalization. Binding was assessed using a liquid scintillation counter.

FIGS. 8A-8C. PSMA expression promotes cell migration. (A) The effect of PSMA expression on cell migration was assessed using a scratch-wound migration assay. Mouse colorectal CT26 (left panels) and human prostate cancer PC-3 (right panels) cell lines with (triangles) or without (squares) stable expression of human PSMA. Representative images are depicted. (B) The effect of PSMA on cell migration was confirmed using a transwell migration assay. CT26 (PSMA−) cells, grey bar; CT26 (PSMA++) cells, black bar. (C) Effect of PSMA small molecule inhibitor (2-PMPA) on migration of PSMA-expressing cells. Without 2-PMPA (triangles), with 2-PMPA (open diamonds). Representative images are depicted. In all panels, *=p<0.05, **=p<0.001.

FIGS. 9A-9B. PSMA expressing cell lines. Cell-surface expression of PSMA in (A) mouse colorectal carcinoma (CT26) cells or (B) human prostate cancer cell line (PC-3) was confirmed by flow cytometry using a PSMA specific antibody. PSMA (−), no PSMA expression; PSMA (+), low PSMA expression; PSMA (++), high PSMA expression.

FIGS. 10A-10B. PSMA expression and proliferation. The effect of PSMA expression on proliferation was assessed using an MTS assay by measuring absorbance (A490). Assessment was performed in (A) a CT26 cell line and (B) a PC-3 cell line, with or without the stable exogenous expression of human PSMA.

FIG. 11. Inhibition of PSMA enzymatic activity on cell membrane extracts. The enzymatic activity of cell membrane extracts from CT26 and PC3 cells in the presence (light gray bars) or absence (black bars) of the small molecule inhibitor 2-PMPA. Values are expressed relative to the NAALADase activity of recombinant PSMA.

FIGS. 12A-12B. PSMA expression promotes cell invasion. (A) The effect of PSMA expression on cell invasion was assessed using a Matrigel-coated transwell invasion assay in both mouse colorectal carcinoma (CT26) and human prostate cancer cells (PC-3) lacking or expressing PSMA. No PSMA expression (−, light grey bars); low PSMA expression (+, dark grey bars); high PSMA expression (++, black bars). (B) Effect of PSMA small molecule inhibitor (2-PMPA) on invasion of PSMA-expressing cells. No PSMA expression (−, light grey bars); low PSMA expression (+, dark grey bars); high PSMA expression (++, black bars). Data represent cells counted from six independent 10× images and error bars depict Standard Error Mean (SEM).

FIGS. 13A-13C. PSMA expression does not affect cell survival. The effect of PSMA expression on cell survival following (A) γ-radiation treatment, (B) UV-C treatment and (C) docetaxel treatment. Survival was assessed using clonogenic survival assays (γ-radiation, UV-C) and MTS assay (docetaxel). Each point is an average of 3 independent experiments. The data were normalized to untreated cells. PC-3 (PSMA−) cells, squares; PC-3 (PSMA+) cells, triangles.

FIGS. 14A-14B. Inhibition of cell-derived PSMA enzymatic activity by synthetic RNA aptamer ligands. (A) Inhibition of PSMA enzymatic activity was evaluated using membrane extracts from PC-3 (PSMA+) cells. Inhibition of PSMA enzymatic activity was evaluated following treatment with the inhibitory PSMA RNA A9g aptamers. A9g (filled squares) inhibited with an IC₅₀=290 nM. A10-3.2 (filled triangles), a PSMA-binding non-inhibitory aptamer and A9g.6 (open squares), a previously described point mutant, non-binding RNA aptamer was used as a negative control in this assay. (B) Inhibition of PSMA enzymatic activity was evaluated using membrane extracts from PC-3 (PSMA +) cells. The full-length non-competitive synthetic RNA aptamer inhibitor (A9; filled circles) inhibited with an IC₅₀=267 nM. A scrambled, non-binding RNA (A9scr; open circles) was used as a negative control in this assay.

FIGS. 15A-15D Inhibition of PSMA-mediated carcinogenesis by synthetic RNA aptamer ligand. (A) Effect of PSMA RNA aptamers on cell migration using a transwell migration assay. A9g (filled squares), A9g.6 (open squares), A10-3.2 (filled triangles), A10-3.2 (filled triangles), 2-PMPA (red circle). The inhibitory A9g aptamer inhibited cell migration with an IC₅₀=18 nM. (B) Effect of PSMA RNA aptamers on cell migration using a transwell migration assay. A9 (filled circles) and A9scr (open circles). The inhibitory A9 aptamer inhibited cell migration with an IC₅₀=81 nM. (C) Dose-response titration of RNA aptamers on cell invasion in transwell invasion assays. A9g (filled squares), A9g.6 (open squares), A10-3.2 (filled triangles), 2-PMPA (red circle). The inhibitory A9g aptamer inhibited cell invasion with an IC₅₀=75 nM (D) Effect of PSMA RNA aptamers on cell invasion using a Matrigel-coated transwell migration assay in cells with (black bars) and without (white bars) PSMA expression. Data represent cells counted from six independent 10× images and error bars depict Standard Error Mean (SEM).

FIGS. 16A-16C. Evaluation of the in vivo efficacy of the A9g aptamer in a metastatic model of prostate cancer. (A) provides representative images of mice treated with either DPBS, A9g or A9g.6, (B) graph showing the data from (A), and (C) provides data regarding tumors excised from mice.

FIGS. 17A-17B. Validation of the Aptamer-Irdye800 CW Conjugate. (A) Percent NAALADase activity. (B) Shows an in vitro comparison of PBS to A9g-IRDye800 and A9g.6-IRDye800.

FIG. 18A-18B. In Vivo Validation of the Aptamer-Irdye800 CW Conjugate. (A) Shows the localization of the A9g-IRdye800CW (2 nmoles) aptamer over time. (B) Shows the localization of the A9g-IRdye800CW (2 nmoles) aptamer over time.

### DETAILED DESCRIPTION OF THE INVENTION

An embodiment of the invention described herein is an optimized RNA-based therapeutic reagent for the treatment of prostate and possibly other solid sarcomas and carcinomas. In certain embodiments, the reagent consists of a single component, an RNA aptamer. In certain embodiments, the reagent consists of two basic components, an RNA aptamer (a structural, synthetic RNA) coupled to a small molecule. The aptamer portion of the reagent serves as a targeting moiety by binding specifically to a cell surface receptor (e.g., prostate specific membrane antigen; PSMA) expressed on cancer cells (e.g., prostate cancer cells).

Aptamer Portion

Aptamers are single stranded oligonucleotides that can naturally fold into different 3-dimensional structures, which have the capability of binding specifically to biosurfaces, a target compound or a moiety. The term “conformational change” refers to the process by which a nucleic acid, such as an aptamer, adopts a different secondary or tertiary structure. The term “fold” may be substituted for conformational change.

Aptamers have advantages over more traditional affinity molecules such as antibodies in that they are very stable, can be easily synthesized, and can be chemically manipulated with relative ease. Aptamer synthesis is potentially far cheaper and reproducible than antibody-based diagnostic tests. Aptamers are produced by solid phase chemical synthesis, an accurate and reproducible process with consistency among production batches. An aptamer can be produced in large quantities by polymerase chain reaction (PCR) and once the sequence is known, can be assembled from individual naturally occurring nucleotides and/or synthetic nucleotides. Aptamers are stable to long-term storage at room temperature, and, if denatured, aptamers can easily be renatured, a feature not shared by antibodies. Furthermore, aptamers have the potential to measure concentrations of ligand in orders of magnitude lower (parts per trillion or even quadrillion) than those antibody-based diagnostic tests. These characteristics of aptamers make them attractive for diagnostic applications.

Aptamers are typically oligonucleotides that may be single stranded oligodeoxynucleotides, oligoribonucleotides, or modified oligodeoxynucleotide or oligoribonucleotides. The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N-6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N-6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine.

The aptamers of the invention are synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R; P(O)OR6; CO; or CONR′2 wherein R is H (or a salt) or alkyl (1-12C) and Rd is alkyl (1-9C) is joined to adjacent nucleotides through —O— or —S—.

In certain embodiments of the present invention, the aptamer portion binds to Prostate-Specific Mediated Antigen (PSMA). In the literature, a PSMA aptamer of 70 nucleotides (A9) was described (Lupold et al., “Identification and characterization of nuclease-stabilized RNA molecules that bind human prostate cancer cells via the prostate-specific membrane antigen Cancer Res. 2002 Jul. 15; 62(14):4029-33). Surprisingly, the inventors were able to shorten the A9 aptamer down to 41 nucleotides (A9L), and still have effective binding activity and inhibitory activity. In certain embodiments, additional modifications are made to the aptamer portion. Additional modifications to the aptamer portion include 2′O-methyl modification of the pyrimidines. In other embodiments, all of the nucleotides in the aptamer are 2′O-methyl modified. Alternatively, the pyrimidines, or all the nucleotides, may be modified with 2′fluoros (both pyrimidines and purines). Additional modifications to the nucleotides in the aptamer include large molecular weight conjugates like pegylation, lipid-based modifications (e.g., cholesterol) or nanoparticles (e.g., PEI or chitosan) to improve the pharmacokinetic/dynamic profile of the chimera.

Prostate-specific membrane antigen (PSMA) is expressed extracellularly on prostate cancer cells (and other solid tumors, such as renal cancer cells) and the endothelial cells of new blood vessels that supply most other solid tumors. However, it has also been shown to be present at low levels in the brain, kidneys (brush border of proximal tubes) and liver. One advantage of targeting PSMA is that it is a transmembrane protein, and is not secreted. The truncated PSMA aptamer can be used as a tool to target prostate cancer as well as the vasculature of all solid sarcomas and carcinomas. It has been previously shown that PSMA expression is elevated in malignant prostate disease as well as tumor vasculature.

In certain embodiments, modifications are introduced into the stem sequence in the aptamer. Different nucleotides can be used as long as the structure of the stem is retained.

Small Molecule Portion

The aptamers of the present invention can be operably linked to one or more small molecule entities. In certain embodiments, the entity is a fluorescent tag, affinity tag, a protein, a solid substrate, a cell surface, or a cellular component. In certain embodiments, the cellular component is a cell wall or cell membrane. In certain embodiments, the solid substrate is a component of silica, cellulose, cellulose acetate, nitrocellulose, nylon, polyester, polyethersulfone, polyolefin, or polyvinylidene fluoride, or combinations thereof. In certain embodiments, the solid substrate is a filter, magnetic bead, metal oxide, latex particle, microtiter plates, polystyrene bead, or CD-ROM.

In certain embodiments, the aptamer is linked to the entity by means of a linker. In certain embodiments, the linker is a binding pair. In certain embodiments, the “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate, IgG-protein A, antigen-antibody, and the like. In certain embodiments, a first member of the binding pair comprises avidin or streptavidin and a second member of the binding pair comprises biotin. In certain embodiments, the aptamer is linked to the entity by means of a covalent bond.

The entity, for example, may additionally or alternatively, be a detection means. A number of “molecular beacons” (such as fluorescence compounds) can be attached to aptamers to provide a means for signaling the presence of and quantifying a target chemical or biological agent. Other exemplary detection labels that could be attached to the aptamers include biotin, any fluorescent dye, amine modification, horseradish peroxidase, alkaline phosphatase, etc.

In certain embodiments, the aptamer is operably linked to a detection means and to a solid substrate. For example, the aptamer may be linked to a fluorescent dye and to a magnetic bead.

The small molecule portion of the ligand can be siRNA sequences, miRNAs, small molecule inhibitors, chelators for housing radionuclides (for diagnostic/imaging applications as well as development of targeted radiotherapies, see, e.g., Rockey et al., Synthesis and radiolabeling of chelator-RNA aptamer bioconjugates with copper-64 for targeted molecular imaging, Bioorganic & Medicinal Chemistry, 19: 4080-4090 (2011)), nanoparticles containing all of the above plus DNA vectors and/or mRNA sequences, depending on the use of the ligand as a diagnostic agent or as a therapeutic agent. In certain embodiments, the small molecule is an RNAi molecule, such as an siRNA or an miRNA. The RNAi portion, upon delivery to the targeted cells, induces the depletion of cancer cell survival factors, leading to the death of the cancer cells. In certain embodiments, the siRNA portion binds to polo-like kinase 1 (Plk1) within the cell, inhibiting the gene's activity. After the aptamer binds PSMA expressed on the surface of the cell, the complex is taken into the cell by endocytosis. The molecule is then cleaved by Dicer, an endonuclease, and is incorporated into the RNA-Induced Silencing Complex (RISC) where it mediates Plk1 degradation.

A first generation of this reagent was previously described in the literature (McNamara et al., Nat Biotechnol. 24(8):1005-15 (2006)). In certain embodiments, the invention encompasses a truncated RNA aptamer, which when compared to the original, longer RNA, is significantly less expensive to produce and an siRNA portion that has been optimized for activity. This optimized reagent surprisingly has a 100-fold greater activity than the first generation reagent when tested in cell culture. These advances result in a cancer therapeutic that is effective at significantly lower doses than the first generation reagent, thus reducing treatment costs as well as the likelihood for toxic side-effects.

Linking Molecules

Chemistries that can be used to link molecules to the aptamer are known in the art, such as disulfide linkages, amino linkages, covalent linkages, etc. Additional linkages and modifications can be found on the world-wide-web at trilinkbiotech.com/products/oligo/oligo_modifications.asp.

Detection and Amplification Methods

The present invention provides methods for detecting PSMA in a sample or in vivo. For example, one can contact a sample with an aptamer as described herein or the composition as described herein to form bound PSMA, and detecting the presence or the quantity of bound PSMA. Alternatively, aptamers or compositions can be administered in vivo to a patient (e.g. injected in situ into a tumor). In certain embodiments, the bound PSMA is detected by means of PCR, nuclear magnetic resonance, fluorescent capillary electrophoresis, lateral flow devices, colorimetry, chemiluminescence, fluorescence, southsester blots, microarrays, or ELISA.

In one embodiment of the present invention, the method also involves contacting the sample with at least one aptamer to form a hybridized nucleic acid and detecting the hybridized nucleic acid. In one embodiment, the detection is by amplification. “Amplifying” utilizes methods such as the polymerase chain reaction (PCR), ligation amplification (or ligase chain reaction, LCR), strand displacement amplification, nucleic acid sequence-based amplification, and amplification methods based on the use of Q-beta replicase. These methods are well known and widely practiced in the art. Reagents and hardware for conducting PCR are commercially available. In one embodiment of the present invention, at least one type of aptamer is immobilized on a solid surface.

The methods of the present invention can be used to detect the presence of PSMA in a sample.

According to the methods of the present invention, the amplification of PSMA present in a sample may be carried out by any means known to the art. Examples of suitable amplification techniques include, but are not limited to, polymerase chain reaction (including, for RNA amplification, reverse-transcriptase polymerase chain reaction), ligase chain reaction, strand displacement amplification, transcription-based amplification, self-sustained sequence replication (or “3SR”), the Qβ replicase system, nucleic acid sequence-based amplification (or “NASBA”), the repair chain reaction (or “RCR”), and boomerang DNA amplification (or “BDA”).

The bases incorporated into the amplification product may be natural or modified bases (modified before or after amplification), and the bases may be selected to optimize subsequent electrochemical detection steps.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized that is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques. Where the nucleic acid to be amplified is RNA, amplification may be carried out by initial conversion to DNA by reverse transcriptase in accordance with known techniques.

Strand displacement amplification (SDA) may be carried out in accordance with known techniques. For example, SDA may be carried out with a single amplification primer or a pair of amplification primers, with exponential amplification being achieved with the latter. In general, SDA amplification primers comprise, in the 5′ to 3′ direction, a flanking sequence (the DNA sequence of which is noncritical), a restriction site for the restriction enzyme employed in the reaction, and an oligonucleotide sequence (e.g., an oligonucleotide probe of the present invention) that hybridizes to the target sequence to be amplified and/or detected. The flanking sequence, which serves to facilitate binding of the restriction enzyme to the recognition site and provides a DNA polymerase priming site after the restriction site has been nicked, is about 15 to 20 nucleotides in length in one embodiment. The restriction site is functional in the SDA reaction. The oligonucleotide probe portion is about 13 to 15 nucleotides in length in one embodiment of the invention.

Ligase chain reaction (LCR) is also carried out in accordance with known techniques. In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

Diagnostic techniques that are useful in the methods of the invention include, but are not limited to direct DNA sequencing, pulsed-field gel electrophoresis (PFGE) analysis, allele-specific oligonucleotide (ASO), dot blot analysis and denaturing gradient gel electrophoresis, and are well known to the artisan.

The sample may be contacted with the aptamer in any suitable manner known to those skilled in the art. For example, the sample may be solubilized in solution, and contacted with the aptamer by solubilizing the aptamer in solution with the sample under conditions that permit binding. Suitable conditions are well known to those skilled in the art. Alternatively, the sample may be solubilized in solution with the aptamer immobilized on a solid support, whereby the sample may be contacted with the aptamer by immersing the solid support having the aptamer immobilized thereon in the solution containing the sample.

General Terminology

“Synthetic” aptamers are those prepared by chemical synthesis. The aptamers may also be produced by recombinant nucleic acid methods. “Recombinant nucleic molecule” is a combination of nucleic sequences that are joined together using recombinant nucleic technology and procedures used to join together nucleic sequences known in the art.

The term “chimeric” refers to a gene or DNA that contains 1) DNA sequences, including regulatory and coding sequences that are not found together in nature or 2) sequences encoding parts of proteins not naturally adjoined, or 3) parts of promoters that are not naturally adjoined. Accordingly, a chimeric gene may include regulatory sequences and coding sequences that are derived from different sources, or include regulatory sequences and coding sequences derived from the same source, but arranged in a manner different from that found in nature.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

By “fragment” or “portion” is meant a full length or less than full length of the nucleotide sequence.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (see the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When using BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the World Wide Web at ncbi.nlm.nih.gov. Alignment may also be performed manually by visual inspection.

For purposes of the present invention, comparison of nucleotide sequences for determination of percent sequence identity to the sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by a BLAST program.

(c) As used herein, “sequence identity” or “identity” in the context of two nucleic acid sequences makes reference to a specified percentage of residues in the two sequences that are the same when aligned for maximum correspondence over a specified comparison window, as measured by sequence comparison algorithms or by visual inspection. When percentage of sequence identity is used in reference to proteins, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code.

(e)(ii) For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched nucleic acid. Specificity is typically the function of post-hybridization washes, the critical factors being the ionic strength and temperature of the final wash solution. For DNA-DNA hybrids, the Tₘ can be approximated from the equation of Meinkoth and Wahl: Tₘ 81.5° C.+16.6 (log M)+0.41 (% GC)−0.61 (% form)−500/L. M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

“Operably-linked” nucleic acids refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule containing less than 31 sequential nucleotides that is transcribed into an RNAi molecule. Such an isolated RNAi molecule may, for example, form a hairpin structure with a duplex 21 base pairs in length that is complementary or hybridizes to a sequence in a gene of interest, and remains stably bound under stringent conditions (as defined by methods well known in the art, e.g., in Sambrook and Russell, 2001). Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

In addition to a DNA sequence encoding a siRNA, the nucleic acid molecules of the invention include double-stranded interfering RNA molecules, which are also useful to inhibit expression of a target gene.

As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

The terms “protein,” “peptide” and “polypeptide” are used interchangeably herein. As used herein, the terms “a” or “an” are used to mean “one or more.”

Modulation of gene expression by endogenous, noncoding RNAs is increasingly appreciated as a mechanism playing a role in eukaryotic development, maintenance of chromatin structure and genomic integrity. Recently, techniques have been developed to trigger RNA interference (RNAi) against specific targets in mammalian cells by introducing exogenously produced or intracellularly expressed siRNAs. These methods have proven to be quick, inexpensive and effective for knockdown experiments in vitro and in vivo. The ability to accomplish selective gene silencing has led to the hypothesis that siRNAs might be employed to suppress gene expression for therapeutic benefit.

Disclosed herein is a strategy that results in substantial silencing of targeted genes via RNAi. Use of this strategy results in markedly diminished in vitro and in vivo expression of targeted genes. This strategy is useful in reducing expression of targeted genes in order to model biological processes or to provide therapy for human diseases. For example, this strategy can be applied to a the treatment of cancer. As used herein the term “substantial silencing” means that the mRNA of the targeted gene is inhibited and/or degraded by the presence of the introduced siRNA, such that expression of the targeted gene is reduced by about 10% to 100% as compared to the level of expression seen when the siRNA is not present. Generally, when a gene is substantially silenced, it will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction expression as compared to when the siRNA is not present. As used herein the term “substantially normal activity” means the level of expression of a gene when an siRNA has not been introduced to a cell.

To accomplish intracellular expression of the therapeutic RNAi molecules, an RNA molecule is constructed containing two complementary strands or a hairpin sequence (such as a 21-bp hairpin) representing sequences directed against the gene of interest. The RNAi molecule, or a nucleic acid encoding the RNAi molecule, is introduced to the target cell, such as a diseased brain cell. The RNAi molecule reduces target mRNA and protein expression.

The construct encoding the therapeutic RNAi molecule is configured such that the one or more strands of the RNAi molecules are encoded by a nucleic acid that is immediately contiguous to a promoter. In one example, the promoter is a pol II promoter. If a pol II promoter is used in a particular construct, it is selected from readily available pol II promoters known in the art, depending on whether regulatable, inducible, tissue or cell-specific expression of the siRNA is desired. The construct is introduced into the target cell, allowing for diminished target-gene expression in the cell.

The present invention provides an expression cassette containing an isolated nucleic acid sequence encoding an RNAi molecule targeted against a gene of interest. The RNAi molecule may form a hairpin structure that contains a duplex structure and a loop structure. The loop structure may be the aptamer portion. The duplex is less than 30 nucleotides in length, such as from 19 to 25 nucleotides. The RNAi molecule may further contain an overhang region. Such an overhang may be a 3′ overhang region or a 5′ overhang region. The overhang region may be, for example, from 1 to 6 nucleotides in length. The expression cassette may further contain a pol II promoter, as described herein. Examples of pal II promoters include regulatable promoters and constitutive promoters. For example, the promoter may be a CMV or RSV promoter. The expression cassette may further contain a polyadenylation signal, such as a synthetic minimal polyadenylation signal. The nucleic acid sequence may further contain a marker gene or stuffer sequences. The expression cassette may be contained in a viral vector. An appropriate viral vector for use in the present invention may be an adenoviral, lentiviral, adeno-associated viral (AAV), poliovirus, herpes simplex virus (HSV) or murine Maloney-based viral vector. The gene of interest may be a gene associated with a condition amenable to siRNA therapy. Examples of such conditions include neurodegenerative diseases, such as a trinucleotide-repeat disease (e.g., polyglutamine repeat disease). Examples of these diseases include Huntington's disease or several spinocerebellar ataxias. Alternatively, the gene of interest may encode a ligand for a chemokine involved in the migration of a cancer cell, or a chemokine receptor.

The present invention also provides an expression cassette containing an isolated nucleic acid sequence encoding a first segment, a second segment located immediately 3′ of the first segment, and a third segment located immediately 3′ of the second segment, wherein the first and third segments are each less than 30 base pairs in length and each more than 10 base pairs in length, and wherein the sequence of the third segment is the complement of the sequence of the first segment, and wherein the isolated nucleic acid sequence functions as an RNAi molecule targeted against a gene of interest. The expression cassette may be contained in a vector, such as a viral vector.

The present invention provides a method of reducing the expression of a gene product in a cell by contacting a cell with an expression cassette described above. It also provides a method of treating a patient by administering to the patient a composition of the expression cassette described above.

The present invention further provides a method of reducing the expression of a gene product in a cell by contacting a cell with an expression cassette containing an isolated nucleic acid sequence encoding a first segment, a second segment located immediately 3′ of the first segment, and a third segment located immediately 3′ of the second segment, wherein the first and third segments are each less than 30 base pairs in length and each more than 10 base pairs in length, and wherein the sequence of the third segment is the complement of the sequence of the first segment, and wherein the isolated nucleic acid sequence functions as an RNAi molecule targeted against a gene of interest.

The present method also provides a method of treating a patient, by administering to the patient a composition containing an expression cassette, wherein the expression cassette contains an isolated nucleic acid sequence encoding a first segment, a second segment located immediately 3′ of the first segment, and a third segment located immediately 3′ of the second segment, wherein the first and third segments are each less than 30 bases in length and each more than 10 bases in length, and wherein the sequence of the third segment is the complement of the sequence of the first segment, and wherein the isolated nucleic acid sequence functions as an RNAi molecule targeted against a gene of interest.

An RNAi molecule may be a “small interfering RNA” or “short interfering RNA” or “siRNA” or “short hairpin RNA” or “shRNA” or “microRNA” or “miRNA.” An RNAi molecule an RNA duplex of nucleotides that is targeted to a nucleic acid sequence of interest. As used herein, the term “RNAi molecule” is a generic term that encompasses the subset of shRNAs. A “RNA duplex” refers to the structure formed by the complementary pairing between two regions of a RNA molecule. RNAi molecule is “targeted” to a gene in that the nucleotide sequence of the duplex portion of the RNAi molecule is complementary to a nucleotide sequence of the targeted gene. In certain embodiments, the RNAi molecules are targeted to the sequence encoding Plk1. In some embodiments, the length of the duplex of RNAi molecules is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the RNAi molecule can be part of a hairpin structure. In addition to the duplex portion, the hairpin structure may contain a loop portion positioned between the two sequences that form the duplex. In certain embodiments, the loop is 9 nucleotides in length. The hairpin structure can also contain 3′ or 5′ overhang portions. In some embodiments, the overhang is a 3′ or a 5′ overhang 0, 1, 2, 3, 4 or 5 nucleotides in length.

A “small interfering” or “short interfering RNA” or siRNA is a RNA duplex of nucleotides that is targeted to a gene interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. The “sense” and “antisense” sequences can be attached to the aptamer portion to form aptamer chimeras. As used herein, the term RNAi molecule is meant to be equivalent to other terms used to describe nucleic acid molecules that are capable of mediating sequence specific RNAi, for example, double-stranded RNA (dsRNA), micro-RNA (miRNA), short hairpin RNA (shRNA), short interfering oligonucleotide, short interfering nucleic acid, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetic silencing. In a non-limiting example, modulation of gene expression by siRNA molecules of the invention can result from siRNA mediated cleavage of RNA (either coding or non-coding RNA) via RISC, or alternately, translational inhibition as is known in the art.

The RNAi molecule can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

The RNAi molecule can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

“Knock-down,” “knock-down technology” refers to a technique of gene silencing in which the expression of a target gene is reduced as compared to the gene expression prior to the introduction of the RNAi molecule, which can lead to the inhibition of production of the target gene product. The term “reduced” is used herein to indicate that the target gene expression is lowered by 1-100%. In other words, the amount of RNA available for translation into a polypeptide or protein is minimized. For example, the amount of protein may be reduced by 10, 20, 30, 40, 50, 60, 70, 80, 90, 95, or 99%. In some embodiments, the expression is reduced by about 90% (i.e., only about 10% of the amount of protein is observed a cell as compared to a cell where RNAi molecules have not been administered). Knock-down of gene expression can be directed, for example, by the use of dsRNAs, siRNAs or miRNAs.

“RNA interference (RNAi)” is the process of sequence-specific, post-transcriptional gene silencing initiated by an RNAi molecule. During RNAi, RNAi molecules induce degradation of target mRNA with consequent sequence-specific inhibition of gene expression. RNAi involving the use of RNAi molecules has been successfully applied to knockdown the expression of specific genes in plants, D. melanogaster, C. elegans, trypanosomes, planaria, hydra, and several vertebrate species including the mouse.

According to a method of the present invention, the expression of PLK1 can be modified via RNAi. For example, the accumulation of PLK1 can be suppressed in a cell. The term “suppressing” refers to the diminution, reduction or elimination in the number or amount of transcripts present in a particular cell. For example, the accumulation of mRNA encoding PLK1 can be suppressed in a cell by RNA interference (RNAi), e.g., the gene is silenced by sequence-specific double-stranded RNA (dsRNA), which is also called short interfering RNA (siRNA). These siRNAs can be two separate RNA molecules that have hybridized together, or they may be a single hairpin wherein two portions of a RNA molecule have hybridized together to form a duplex.

The terms “protein,” “peptide” and “polypeptide” are used interchangeably herein.

“Recombinant DNA molecule” is a combination of DNA sequences that are joined together using recombinant DNA technology and procedures used to join together DNA sequences as described, for example, in Sambrook and Russell (2001).

The terms “heterologous gene,” “heterologous DNA sequence,” “exogenous DNA sequence,” “heterologous RNA sequence,” “exogenous RNA sequence” or “heterologous nucleic acid” each refer to a sequence that either originates from a source foreign to the particular host cell, or is from the same source but is modified from its original or native form. Thus, a heterologous gene in a host cell includes a gene that is endogenous to the particular host cell but has been modified through, for example, the use of DNA shuffling. The terms also include non-naturally occurring multiple copies of a naturally occurring DNA or RNA sequence. Thus, the terms refer to a DNA or RNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA or RNA sequence is a sequence that is naturally associated with a host cell into which it is introduced.

“Genome” refers to the complete genetic material of an organism.

A “vector” is defined to include, inter alia, any viral vector, as well as any plasmid, cosmid, phage or binary vector in double or single stranded linear or circular form that may or may not be self transmissible or mobilizable, and that can transform prokaryotic or eukaryotic host either by integration into the cellular genome or exist extrachromosomally (e.g., autonomous replicating plasmid with an origin of replication).

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. The coding region usually codes for a functional RNA of interest, for example an RNAi molecule. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence. It may constitute an “uninterrupted coding sequence”, i.e., lacking an intron, such as in a cDNA, or it may include one or more introns bounded by appropriate splice junctions. An “intron” is a sequence of RNA that is contained in the primary transcript but is removed through cleavage and re-ligation of the RNA within the cell to create the mature mRNA that can be translated into a protein.

The terms “initiation codon” and “termination codon” refer to a unit of three adjacent nucleotides (a ‘codon’) in a coding sequence that specifies initiation and chain termination, respectively, of protein synthesis (mRNA translation).

“Functional RNA” refers to sense RNA, antisense RNA, ribozyme RNA, siRNA, or other RNA that may not be translated but yet has an effect on at least one cellular process.

The term “RNA transcript” or “transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” are nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

“3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. Examples of promoters that may be used in the present invention include the mouse U6 RNA promoters, synthetic human H1RNA promoters, SV40, CMV, RSV, RNA polymerase II and RNA polymerase III promoters.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of siRNA constructs, expression may refer to the transcription of the siRNA only. In addition, expression refers to the transcription and stable accumulation of sense (mRNA) or functional RNA. Expression may also refer to the production of protein.

“Altered levels” refers to the level of expression in transgenic cells or organisms that differs from that of normal or untransformed cells or organisms.

“Overexpression” refers to the level of expression in transgenic cells or organisms that exceeds levels of expression in normal or untransformed cells or organisms.

“Antisense inhibition” refers to the production of antisense RNA transcripts capable of suppressing the expression of protein from an endogenous gene or a transgene.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule. An example of a cis-acting sequence on the replicon is the viral replication origin.

The terms “trans-acting sequence” and “trans-acting element” refer to DNA or RNA sequences whose function does not require them to be on the same molecule.

“Chromosomally-integrated” refers to the integration of a foreign gene or nucleic acid construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells.

“Transformed,” “transduced,” “transgenic” and “recombinant” refer to a host cell into which a heterologous nucleic acid molecule has been introduced. As used herein the term “transfection” refers to the delivery of DNA into eukaryotic (e.g., mammalian) cells. The term “transformation” is used herein to refer to delivery of DNA into prokaryotic (e.g., E. coli) cells. The term “transduction” is used herein to refer to infecting cells with viral particles. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

“Treating” as used herein refers to ameliorating at least one symptom of, curing and/or preventing the development of a disease or a condition.

Expression Cassettes of the Invention

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

Aside from recombinant DNA sequences that serve as transcription units for an RNA transcript, or portions thereof; a portion of the recombinant DNA may be untranscribed, serving a regulatory or a structural function. For example, the recombinant DNA may have a promoter that is active in mammalian cells.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the siRNA, or the like. Such elements may be included in the DNA as desired to obtain the optimal performance of the siRNA in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

Operably linked nucleic acids are nucleic acids placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as two and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

The general methods for constructing recombinant DNA that can transfect target cells are well known to those skilled in the art, and the same compositions and methods of construction may be utilized to produce the DNA useful herein.

The recombinant DNA can be readily introduced into the host cells, e.g., mammalian, bacterial, yeast or insect cells by transfection with an expression vector composed of DNA encoding the siRNA by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described herein below, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As discussed above, a “transfected” or “transduced” host cell or cell line is one in which the genome has been altered or augmented by the presence of at least one heterologous or recombinant nucleic acid sequence. The host cells of the present invention are typically produced by transfection with a DNA sequence in a plasmid expression vector, a viral expression vector, or as an isolated linear DNA sequence. The transfected DNA can become a chromosomally integrated recombinant DNA sequence, which is composed of sequence encoding the siRNA.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the preselected DNA segment is being expressed. Expression may be evaluated by specifically identifying the peptide products of the introduced recombinant DNA sequences or evaluating the phenotypic changes brought about by the expression of the introduced recombinant DNA segment in the host cell.

According to one embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent and the therapeutic agent is delivered in situ.

Methods for Introducing the Expression Cassettes of the Invention into Cells

The condition amenable to gene inhibition therapy may be a prophylactic process, i.e., a process for preventing disease or an undesired medical condition. Thus, the instant invention embraces a system for delivering siRNA that has a prophylactic function (i.e., a prophylactic agent) to the mammalian recipient.

The inhibitory nucleic acid material (e.g., an expression cassette encoding siRNA directed to a gene of interest) can be introduced into the cell ex vivo or in vivo by genetic transfer methods, such as transfection or transduction, to provide a genetically modified cell. Various expression vectors (i.e., vehicles for facilitating delivery of exogenous nucleic acid into a target cell) are known to one of ordinary skill in the art.

As used herein, “transfection of cells” refers to the acquisition by a cell of new nucleic acid material by incorporation of added DNA. Thus, transfection refers to the insertion of nucleic acid into a cell using physical or chemical methods. Several transfection techniques are known to those of ordinary skill in the art including calcium phosphate DNA co-precipitation, DEAE-dextran, electroporation, cationic liposome-mediated transfection, tungsten particle-facilitated microparticle bombardment, and strontium phosphate DNA co-precipitation.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

The exogenous nucleic acid material can include the nucleic acid encoding the siRNA together with a promoter to control transcription. The promoter characteristically has a specific nucleotide sequence necessary to initiate transcription. The exogenous nucleic acid material may further include additional sequences (i.e., enhancers) required to obtain the desired gene transcription activity. For the purpose of this discussion an “enhancer” is simply any non-translated DNA sequence that works with the coding sequence (in cis) to change the basal transcription level dictated by the promoter. The exogenous nucleic acid material may be introduced into the cell genome immediately downstream from the promoter so that the promoter and coding sequence are operatively linked so as to permit transcription of the coding sequence. An expression vector can include an exogenous promoter element to control transcription of the inserted exogenous gene. Such exogenous promoters include both constitutive and regulatable promoters.

Naturally-occurring constitutive promoters control the expression of essential cell functions. As a result, a nucleic acid sequence under the control of a constitutive promoter is expressed under all conditions of cell growth. Constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the beta-actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others.

Nucleic acid sequences that are under the control of regulatable promoters are expressed only or to a greater or lesser degree in the presence of an inducing or repressing agent, (e.g., transcription under control of the metallothionein promoter is greatly increased in presence of certain metal ions). Regulatable promoters include responsive elements (REs) that stimulate transcription when their inducing factors are bound. For example, there are REs for serum factors, steroid hormones, retinoic acid, cyclic AMP, and tetracycline and doxycycline. Promoters containing a particular RE can be chosen in order to obtain an regulatable response and in some cases, the RE itself may be attached to a different promoter, thereby conferring regulatability to the encoded nucleic acid sequence. Thus, by selecting the appropriate promoter (constitutive versus regulatable; strong versus weak), it is possible to control both the existence and level of expression of a nucleic acid sequence in the genetically modified cell. If the nucleic acid sequence is under the control of an regulatable promoter, delivery of the therapeutic agent in situ is triggered by exposing the genetically modified cell in situ to conditions for permitting transcription of the nucleic acid sequence, e.g., by intraperitoneal injection of specific inducers of the regulatable promoters which control transcription of the agent. For example, in situ expression of a nucleic acid sequence under the control of the metallothionein promoter in genetically modified cells is enhanced by contacting the genetically modified cells with a solution containing the appropriate (i.e., inducing) metal ions in situ.

Accordingly, the amount of siRNA generated in situ is regulated by controlling such factors as the nature of the promoter used to direct transcription of the nucleic acid sequence, (i.e., whether the promoter is constitutive or regulatable, strong or weak) and the number of copies of the exogenous nucleic acid sequence encoding a siRNA sequence that are in the cell.

In one embodiment of the present invention, an expression cassette may contain a poi II promoter that is operably linked to a nucleic acid sequence encoding a siRNA. Thus, the pol II promoter, i.e., a RNA polymerase II dependent promoter, initiates the transcription of the siRNA. In another embodiment, the pol II promoter is regulatable.

A pol II promoter may be used in its entirety, or a portion or fragment of the promoter sequence may be used in which the portion maintains the promoter activity. As discussed herein, pol II promoters are known to a skilled person in the art and include the promoter of any protein-encoding gene, e.g., an endogenously regulated gene or a constitutively expressed gene. For example, the promoters of genes regulated by cellular physiological events, e.g., heat shock, oxygen levels and/or carbon monoxide levels, e.g., in hypoxia, may be used in the expression cassettes of the invention. In addition, the promoter of any gene regulated by the presence of a pharmacological agent, e.g., tetracycline and derivatives thereof, as well as heavy metal ions and hormones may be employed in the expression cassettes of the invention. In an embodiment of the invention, the pol II promoter can be the CMV promoter or the RSV promoter. In another embodiment, the pol II promoter is the CMV promoter.

As discussed above, a pol II promoter of the invention may be one naturally associated with an endogenously regulated gene or sequence, as may be obtained by isolating the 5′ non-coding sequences located upstream of the coding segment and/or exon. The pol II promoter of the expression cassette can be, for example, the same pal II promoter driving expression of the targeted gene of interest. Alternatively, the nucleic acid sequence encoding the RNAi molecule may be placed under the control of a recombinant or heterologous pol II promoter, which refers to a promoter that is not normally associated with the targeted gene's natural environment. Such promoters include promoters isolated from any eukaryotic cell, and promoters not “naturally occurring,” i.e., containing different elements of different transcriptional regulatory regions, and/or mutations that alter expression. In addition to producing nucleic acid sequences of promoters synthetically, sequences may be produced using recombinant cloning and/or nucleic acid amplification technology, including PCR, in connection with the compositions disclosed herein.

In one embodiment, a pol II promoter that effectively directs the expression of the siRNA in the cell type, organelle, and organism chosen for expression will be employed. Those of ordinary skill in the art of molecular biology generally know the use of promoters for protein expression. The promoters employed may be constitutive, tissue-specific, inducible, and/or useful under the appropriate conditions to direct high level expression of the introduced DNA segment, such as is advantageous in the large-scale production of recombinant proteins and/or peptides. The identity of tissue-specific promoters, as well as assays to characterize their activity, is well known to those of ordinary skill in the art.

In addition to at least one promoter and at least one heterologous nucleic acid sequence encoding the siRNA, the expression vector may include a selection gene, for example, a neomycin resistance gene, for facilitating selection of cells that have been transfected or transduced with the expression vector.

Cells can also be transfected with two or more expression vectors, at least one vector containing the nucleic acid sequence(s) encoding the siRNA(s), the other vector containing a selection gene. The selection of a suitable promoter, enhancer, selection gene and/or signal sequence is deemed to be within the scope of one of ordinary skill in the art without undue experimentation.

The following discussion is directed to various utilities of the instant invention. For example, the instant invention has utility as an expression system suitable for silencing the expression of gene(s) of interest.

The instant invention also provides methods for genetically modifying cells of a mammalian recipient in vivo. According to one embodiment, the method comprises introducing an expression vector for expressing a siRNA sequence in cells of the mammalian recipient in situ by, for example, injecting the vector into the recipient.

Thus, as will be apparent to one of ordinary skill in the art, a variety of suitable viral expression vectors are available for transferring exogenous nucleic acid material into cells. The selection of an appropriate expression vector to express a therapeutic agent for a particular condition amenable to gene silencing therapy and the optimization of the conditions for insertion of the selected expression vector into the cell, are within the scope of one of ordinary skill in the art without the need for undue experimentation.

In another embodiment, the expression vector is in the form of a plasmid, which is transferred into the target cells by one of a variety of methods: physical (e.g., microinjection, electroporation, scrape loading, microparticle bombardment) or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (ProMega, Madison, Wis.). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation.

Diseases and Conditions Amendable to the Methods of the Invention

In the certain embodiments of the present invention, a mammalian recipient to an expression cassette of the invention has a condition that is amenable to gene silencing therapy. As used herein, “gene silencing therapy” refers to administration to the recipient exogenous nucleic acid material encoding a therapeutic siRNA and subsequent expression of the administered nucleic acid material in situ. Thus, the phrase “condition amenable to siRNA therapy” embraces conditions such as genetic diseases (i.e., a disease condition that is attributable to one or more gene defects), acquired pathologies (i.e., a pathological condition that is not attributable to an inborn defect), cancers, neurodegenerative diseases, e.g., trinucleotide repeat disorders, and prophylactic processes (i.e., prevention of a disease or of an undesired medical condition). A gene “associated with a condition” is a gene that is either the cause, or is part of the cause, of the condition to be treated. Examples of such genes include genes associated with a neurodegenerative disease (e.g., a trinucleotide-repeat disease such as a disease associated with polyglutamine repeats, Huntington's disease, and several spinocerebellar ataxias), and genes encoding ligands for chemokines involved in the migration of a cancer cells, or chemokine receptor. Also siRNA expressed from viral vectors may be used for in vivo antiviral therapy using the vector systems described.

Accordingly, as used herein, the term “therapeutic siRNA” refers to any siRNA that has a beneficial effect on the recipient. Thus, “therapeutic siRNA” embraces both therapeutic and prophylactic siRNA.

Differences between alleles that are amenable to targeting by siRNA include disease-causing mutations as well as polymorphisms that are not themselves mutations, but may be linked to a mutation or associated with a predisposition to a disease state.

A condition amenable to gene silencing therapy can be a genetic disorder or an acquired pathology that is manifested by abnormal cell proliferation, e.g., cancer. According to this embodiment, the instant invention is useful for silencing a gene involved in neoplastic activity. The present invention can also be used to inhibit overexpression of one or several genes. The present invention can be used to treat neuroblastoma, medulloblastoma, or glioblastoma.

Dosages, Formulations and Routes of Administration of the Agents of the Invention

The agents of the invention are preferably administered so as to result in a reduction in at least one symptom associated with a disease. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems, which are well known to the art.

Administration of the aptamer chimera may be accomplished through the administration of the nucleic acid molecule. Pharmaceutical formulations, dosages and routes of administration for nucleic acids are generally known in the art.

The present invention envisions treating a disease, for example, cancer, in a mammal by the administration of an agent, e.g., a nucleic acid composition, an expression vector, or a viral particle of the invention. Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms having the therapeutic agent(s) of the invention, which, as discussed below, may optionally be formulated for sustained release (for example using microencapsulation), can be administered by a variety of routes including parenteral, including by intravenous and intramuscular routes, as well as by direct injection into the diseased tissue. For example, the therapeutic agent may be directly injected into the cancer. In another example, the therapeutic agent may be introduced intramuscularly for viruses that traffic back to affected neurons from muscle, such as AAV, lentivirus and adenovirus. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules, as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0, saline solutions, and water.

### Example 1

Rational Truncation of an RNA Aptamer to Prostate Specific Membrane Antigen Using Computational Structural Modeling

RNA aptamers represent an emerging class of pharmaceuticals with great potential for targeted cancer diagnostics and therapy. Several RNA aptamers that bind cancer cell-surface antigens with high affinity and specificity have been described. However, their clinical potential has yet to be realized. A significant obstacle to the clinical adoption of RNA aptamers is the high cost of manufacturing long RNA sequences through chemical synthesis. Therapeutic aptamers are often truncated post-selection using a trial-and-error process, which is time consuming and inefficient. Here we used a “rational truncation” approach guided by RNA structural prediction and protein/RNA docking algorithms that enabled us to substantially truncate A9, a RNA aptamer to prostate specific membrane antigen (PSMA), with great potential for targeted therapeutics. This truncated PSMA aptamer (A9L; 41 mer) retains binding activity, functionality, and is amenable to large-scale chemical synthesis for future clinical applications. In addition, the RNA tertiary structure and protein/RNA docking predictions revealed key nucleotides within the aptamer critical for binding to PSMA and inhibiting its enzymatic activity. Finally, this work highlights the utility of existing RNA structural prediction and protein docking techniques that may be generally applicable to developing optimized RNA aptamers for therapeutic use.

RNA aptamers are synthetic, single-stranded oligonucleotide ligands typically 30 to 70 bases in length, which adopt complex three-dimensional conformations to bind targets with high affinity and specificity. The targets of RNA aptamers include small molecules, peptides, proteins (secreted factors, intracellular proteins and membrane receptors), and even whole cells. High affinity RNA aptamers for specific targets can be derived from combinatorial RNA sequence libraries (with complexities of ˜10¹⁴) by an iterative selection process termed SELEX (Systematic Evolution of Ligands by EXponential Enrichment). To enable the use of RNA aptamers for in vivo applications, modified nucleotides (e.g. 2′-fluoro pyrimidines, 2′-amino pyrimidines, or 2′-O-methyl ribose purines and pyrimidines are usually incorporated during the selection process or post-selection during chemical synthesis.

The affinities and specificities of RNA aptamers for their targets are comparable to those of antibodies for their antigens. Like antibodies, RNA aptamers can be used for targeted diagnostics and therapeutics. At the bench, RNA aptamers have been successfully used as inhibitors of their targets as well as to deliver chemotherapeutic agents, nanoparticles, radionuclides, and siRNAs to specific cell-types in culture and in vivo. Several RNA aptamers are currently undergoing clinical trials and one, Pegaptanib, was approved for therapeutic use in age-related macular degeneration by the US Food and Drug Administration in 2004. As targeted therapeutic agents, RNA aptamers have several advantages over antibodies, such as smaller size, better tissue penetration, ease of chemical synthesis/modification and the lack of immune stimulation. Furthermore, from the standpoint of pharmaceutical manufacturing, RNA aptamers are not classified as biological agents thus easing regulatory approval.

Despite these advantages, a current obstacle to delivering RNA aptamer technology to the clinic cost-effectively is the ability to chemically synthesize long RNAs (>60 nucleotides) in large-scale quantities. Aptamer production is based on solid-phase phosphoroamidite chemistry via an automated process used for small-scale oligonucleotide synthesis. This process is highly reproducible allowing short synthetic RNA aptamers (15-50 nucleotides in length) to be purified to a high degree of purity/stability and synthetic yield. However, RNA aptamers of long length remain difficult to synthesize under these conditions. Although the efficiency of the manufacturing process for synthetic oligonucleotides continues to improve, perhaps the simplest way to ensure high synthetic yield is to decrease the length of the oligonucleotide sequence to be synthesized. One potential solution to this problem is the identification of shorter RNA aptamer sequences through the use of short RNA SELEX libraries (less than 50 nucleotides in length). However, the downside to this approach is a reduction in the sequence complexity of the overall RNA aptamer library which could compromise the identification of optimal sequences.

A more common method to reducing the length of RNA aptamers has been extensive truncation of aptamer sequences post-selection using a trial-and-error approach which is often time-consuming and arduous, and is not guaranteed to work for all aptamers. A key example of this has been the truncation of RNA aptamers that bind to prostate specific membrane antigen (PSMA). The trial-and-error approach was used successfully by Lupold and colleagues to truncate one of two nuclease-resistant RNA aptamers (A9 and A10) which were selected to inhibit PSMA enzymatic activity (Lupold, et al., (2002) Identification and characterization of nuclease-stabilized RNA molecules that bind human prostate cancer cells via the prostate-specific membrane antigen. Cancer Res, 62, 4029-4033)). By consecutively removing 5 bases from the 3′-terminus the authors were able to truncate the A10 RNA aptamer from 71 to 56 nucleotides (A10-3) while retaining functionality (ability to inhibit PSMA enzymatic activity) and ability to be in vitro transcribed using a T7 RNA polymerase. However, when a similar truncation approach was applied to the A9 aptamer in this study, the aptamer was rendered inactive.

Given the therapeutic potential of the PSMA RNA aptamers for applications including inhibition of PSMA's pro-carcinogenic properties and delivery of small molecule drugs/toxins, therapeutic siRNAs, and nanoparticles to prostate cancer cells, further optimization to facilitate large-scale chemical synthesis of these RNAs is compelling. Toward this end, we have employed computational RNA structural modeling and RNA/protein docking models to guide the truncation of the A9 PSMA RNA aptamer. This analysis resulted in a truncated derivative of the A9 aptamer (A9L, 41mer) which, due to its reduced length, is now amenable to large-scale chemical synthesis. Importantly, A9L retains PSMA binding activity/specificity and functionality. Specifically, we show that A9L inhibits PSMA's enzymatic activity and when directly applied to cells expressing PSMA, is effectively internalized.

In summary, these studies demonstrate the utility of computational RNA secondary and tertiary structure models for guiding/enabling truncations of RNA aptamers while retaining their function. Furthermore, these studies have resulted in versions of the PSMA A9 aptamer that due to their shorter sequence length are now amenable to large-scale chemical synthesis for therapeutic applications.

### Materials & Methods

DNA Templates and primers for generating the duplex DNA used for transcription of the RNA aptamers:

A9a aptamer
DNA Template: 5′-
GGGAGGACGATGCGGACCGAAAAAGACCTGACTTCTATACTAAGTCTA
CGTTCCCAGACGACTCCC -3′
5′ primer:
5′-TAATACGACTCACTATAGGGAGGACGATGCGGA-3′
3′ primer:
5′-GGGAGTCGTCTGGGAA-3′
A9b aptamer
DNA Template: 5′-
GGGACGATGCGGACCGAAAAAGACCTGACTTCTATACTAAGTCTACGT
TCCCAGACGCCC-3′
5′ primer:
5′-TAATACGACTCACTATAGGGACGATGCGGACCG-3′
3′ primer:
5′-GGGCGTCTGGGAACGT-3′
A9c aptamer
DNA Template: 5′-
GGGATGCGGACCGAAAAAGACCTGACTTCTATACTAAGTCTACGTTCC
CAGACCC-3′
5′ primer:
5′- TAATACGACTCACTATAGGGATGCGGACCGAAA-3′
3′ primer:
5′-GGGTCTGGGAACGTAG-3′
A9d aptamer
DNA Template: 5′-
GGGACGATGCGGACCGAAAAAGACCTGACTTCTATACTAAGTCTACGT
TCCCAGACGACCC-3′
5′ primer:
5′-TAATACGACTCACTATAGGGACGATGCGGACCG-3′
3′ primer:
5′-GGGTCGTCTGGGAACG-3′
A9e aptamer
DNA Template: 5′-
GGGCGGACCGAAAAAGACCTGACTTCTATACTAAGTCTACGTTCCCAC
C-3′
5′ primer:
5′-TAATACGACTCACTATAGGGCGGACCGAAAAAG-3′
3′ primer:
5′-GGTGGGAACGTAGACT-3′
A9f aptamer
DNA Template: 5′-
GGGCGGACCGAAAAAGACCTGACTTCTATACTAAGTCTACGTTCCCAG
CCC-3′
5′ primer:
5′-TAATACGACTCACTATAGGGCGGACCGAAAAAG-3′
3′ primer:
5′-GGGCTGGGAACGTAGA-3′
A9g aptamer
DNA Template: 5′-
GGGACCGAAAAAGACCTGACTTCTATACTAAGTCTACGTTCCC-3′
5′primer:
5′-TAATACGACTCACTATAGGGACCGAAAAAGACC -3′
3′ primer:
5′-GGGAACGTAGACTTAG-3′

Chemically Synthesized Double Stranded DNA Templates Used for Transcription of the RNA Aptamers:

A9g aptamer
Sense: 5′-
TAATACGACTCACTATAGGGACCGAAAAAGACCTGACTTCTATACTAAG
TCTAC GTTCCC-3′
Antisense: 5′-
GGGAACGTAGACTTAGTATAGAAGTCAGGTCTTTTTCGGTCCCTATAGTG
A GTCGTATTA -3′
A9h aptamer
Sense: 5′-
TAATACGACTCACTATAGGGGAAAAAGACCTGACTTCTATACTAAGTCT
ACCCC-3′
Antisense: 5′-
GGGGTAGACTTAGTATAGAAGTCAGGTCTTTTTCCCCTATAGTGAGTCGT
A TTA -3′
A9i aptamer
Sense: 5′-
TAATACGACTCACTATAGGGCCTGACTTCTATACTAAGCCC-3′
Antisense: 5′-
GGGCTTAGTATAGAAGTCAGGCCCTATAGTGAGTCGTATTA-3′
A9i aptamer
Sense: 5′-
TAATACGACTCACTATAGGGACCGAAAAAGACCTAGTCTACGTTCCC-3′
Antisense: 5′-
GGGAACGTAGACTAGGTCTTTTTCGGTCCCTATAGTGAGTCGTATTA-3′
A9k aptamer
Sense: 5′-
TAATACGACTCACTATAGGGACCGAAAAATACGTTCCC-3′
Antisense: 5′-
GGGAACGTATTTTTCGGTCCCTATAGTGAGTCGTATTA-3′
A9L aptamer
Sense: 5′-
TAATACGACTCACTATAGGGCCGAAAAAGACCTGACTTCTATACTAAGT
CTACG TCCC-3′
Antisense: 5′-
GGGACGTAGACTTAGTATAGAAGTCAGGTCTTTTTCGGCCCTATAGTGAG
T CGTATTA-3′
A9g.1 aptamer
Sense: 5′-
TAATACGACTCACTATAGGGACCGAAAAAGGCCTGACTTCTATACTAAG
CCTAC GTTCCC-3′
Antisense: 5′-
GGGAACGTAGGCTTAGTATAGAAGTCAGGCCTTTTTCGGTCCCTATAGTG
A GTCGTATTA-3′
A9g.2 aptamer
Sense: 5′-
TAATACGACTCACTATAGGGACCGAAAAAGCCCTGACTTCTATACTAAG
GCTAC GTTCCC-3′
Antisense: 5′-
GGGAACGTAGCCTTAGTATAGAAGTCAGGGCTTTTTCGGTCCCTATAGTG
A GTCGTATTA-3′
A9g.3 aptamer
Sense: 5′-
TAATACGACTCACTATAGGGACCGAAAAAGACCTGACTTCTATACTAAG
TCTAC GGTCCC-3′
Antisense: 5′-
GGGACCGTAGACTTAGTATAGAAGTCAGGTCTTTTTCGGTCCCTATAGTG
A GTCGTATTA-3′
A9g.4 aptamer
Sense: 5′-
TAATACGACTCACTATAGGGACCGAAAAAGACCTGACTTCTATACTAAG
TCTTC GTTCCC-3′
Antisense: 5′-
GGGAACGAAGACTTAGTATAGAAGTCAGGTCTTTTTCGGTCCCTATAGTG
A GTCGTATTA -3′
A9g.5 aptamer
Sense: 5′-
TAATACGACTCACTATAGGGACCGAAAAAGACCTGACTTCTATACTAGG
TCTAC GTTCCC-3′
Antisense: 5′-
GGGAACGTAGACCTAGTATAGAAGTCAGGTCTTTTTCGGTCCCTATAGTG
A GTCGTATTA-3′
A9g.6 aptamer
Sense: 5′-
TAATACGACTCACTATAGGGACCGAAAAAGACCTGGCTTCTATACTAAG
TCTAC GTTCCC-3′
Antisense: 5′-
GGGAACGTAGACTTAGTATAGAAGCCAGGTCTTTTTCGGTCCCTATAGTG
A GTCGTATTA-3′
A9g.7 aptamer
Sense: 5′-
TAATACGACTCACTATAGGGACCGAAAAAGACCTGACTTCTATACTAAG
TCTAC GATCCC-3′
Antisense: 5′-
GGGATCGTAGACTTAGTATAGAAGTCAGGTCTTTTTCGGTCCCTATAGTG
A GTCGTATTA-3′
A9g.8 aptamer
Sense: 5′-
TAATACGACTCACTATAGGGACCGAAAAAGACCTGACTTCTATACTAAG
TCTAC GCTCCC-3′
Antisense: 5′-
GGGAGCGTAGACTTAGTATAGAAGTCAGGTCTTTTTCGGTCCCTATAGTG
A GTCGTATTA-3′

| | SEQ | | page | |
|---|---|---|---|---|
| | ID | | in | |
| Name | NO | Sequence | spec. | length |
| Generic | 1 | 5′-N₁GGRCCGAMAAAGVCCTGACTTCTATACTAAGBCTWCGYYCCN₂-3′ | | 41 |
| sequence | | | | |
| A9a | 2 | 5′-GGGAGGACGATGCGGACCGAAAAAGACCTGACTTCTATACTAAGTCTACGTTCCCAGACGACTCCC- | | 66 |
| | | 3′ | | |
| A9b | 3 | 5′-GGGACGATGCGGACCGAAAAAGACCTGACTTCTATACTAAGTCTACGTTCCCAGACGCCC-3′ | | 60 |
| A9c | 4 | 5′-GGGATGCGGACCGAAAAAGACCTGACTTCTATACTAAGTCTACGTTCCCAGACCC-3′ | | 55 |
| A9d | 5 | 5′-GGGACGATGCGGACCGAAAAAGACCTGACTTCTATACTAAGTCTACGTTCCCAGACGACCC-3′ | | 61 |
| A9e | 6 | 5′-GGGCGGACCGAAAAAGACCTGACTTCTATACTAAGTCTACGTTCCCACC-3′ | | 49 |
| A9f | 7 | 5′-GGGCGGACCGAAAAAGACCTGACTTCTATACTAAGTCTACGTTCCCAGCCC-3′ | | 51 |
| A9g | 8 | 5′-GGGACCGAAAAAGACCTGACTTCTATACTAAGTCTACGTTCCC-3′ | | 43 |
| A9g.1 | 9 | 5′-GGGACCGAAAAAGGCCTGACTTCTATACTAAGCCTACGTTCCC-3′ | | 43 |
| A9g.2 | 10 | 5′-GGGACCGAAAAAGCCCTGACTTCTATACTAAGGCTACGTTCCC-3′ | | 43 |
| A9g.4 | 11 | 5′-GGGACCGAAAAAGACCTGACTTCTATACTAAGTCTTCGTTCCC-3′ | | 43 |
| A9g.9/A9C | 12 | 5′-GGGACCGAAAAAGACCTGACTTCTATACTAAGTCTACGTTCCC-3′ | | 43 |
| A9L | 13 | 5′-GGGCCGAAAAAGACCTGACTTCTATACTAAGTCTACGTCCC-3′ | | 41 |

RNA Truncations

To generate the A9 truncations, the sequence of full-length A9 as previously reported (Lupoid, et al., (2002) Identification and characterization of nuclease-stabilized RNA molecules that bind human prostate cancer cells via the prostate-specific membrane antigen. Cancer Res, 62, 4029-4033) (5′-GGGAGGACGAUGCGGACCGAAAAAGACCUGACUUCUAUACUA AGUCUACGUUCCCAGACGACUCGCCCGA-3′) was loaded into the program RNAStructure 4.6 (Mathews, et al., (2007) RNA secondary structure prediction. Curr Protoc Nucleic Acid Chem, Chapter 11, Unit 11 12; Mathews, D. H. (2006) RNA secondary structure analysis using RNAstructure. Curr Protoc Bioinformatics, Chapter 12, Unit 12 16). Using a computer-guided “rational truncation” approach, bases were removed from the 5′ and 3′ ends such that the predicted secondary structure of the remaining oligonucleotide was as similar as possible to that of full-length A9. Where necessary, base changes were made at the 5′ and 3′ ends to maintain a 5′-GGG transcription start codon and a complementary 3′-CCC. To create the illustrations, the secondary structures were rendered with the program VARNA 3.7 (Darty, K., et al., (2009) VARNA: Interactive drawing and editing of the RNA secondary structure. Bioinformatics, 25, 1974-1975).

RNA Transcriptions

The RNA was transcribed as previously described (McNamara, J. O., 2nd, et al., (2006) Cell type-specific delivery of siRNAs with aptamer-siRNA chimeras. Nat Biotechnol, 24, 1005-1015). Briefly, template DNAs and primers were ordered from IDT (Coralville, Iowa). Using the above primer and template sequences, the double-stranded DNA templates for transcription were generated. DNA templates were purified with Qiagen DNA purification columns (27106) and used in in vitro transcription reactions as described in McNamara et al., 2006 to make individual RNA aptamers. A Y639F mutant T7 RNA polymerase (Huang, Y., et al., (1997) Mechanism of ribose 2′-group discrimination by an RNA polymerase. Biochemistry, 36, 8231-8242) was used to incorporate 2′ fluoro modified pyrimidines to render the RNAs resistant to nuclease degradation. The RNA from the transcription was run on a denaturing 10% acrylamide/7M urea gel, visualized using UV shadowing. The RNA was excised from the gel, eluted in 4 mL of TE buffer, washed twice with 4 mL of TE buffer and concentrated with an Amicon 10,000 MW-cutoff spin filter (UFC801024).

As an alternative to amplifying the double-stranded DNA templates by PCR, the complete sense and antisense strands of the RNA transcription template were ordered from IDT. To anneal the two, each oligonucleotide strand was added to 500 μL of PCR-grade H₂O to a final concentration of 3 μM per strand, heated to 72° C. for 5 minutes, and then allowed to cool to room temperature over 10 minutes. The resulting double-stranded DNA was used in an RNA transcription reaction as described above. The aptamers A9g, A9h, A9i, A9j, A9k, A9L, and all aptamer mutations were transcribed from chemically synthesized double-stranded DNA templates in this fashion.

PSMA NAALADase Activity Assay

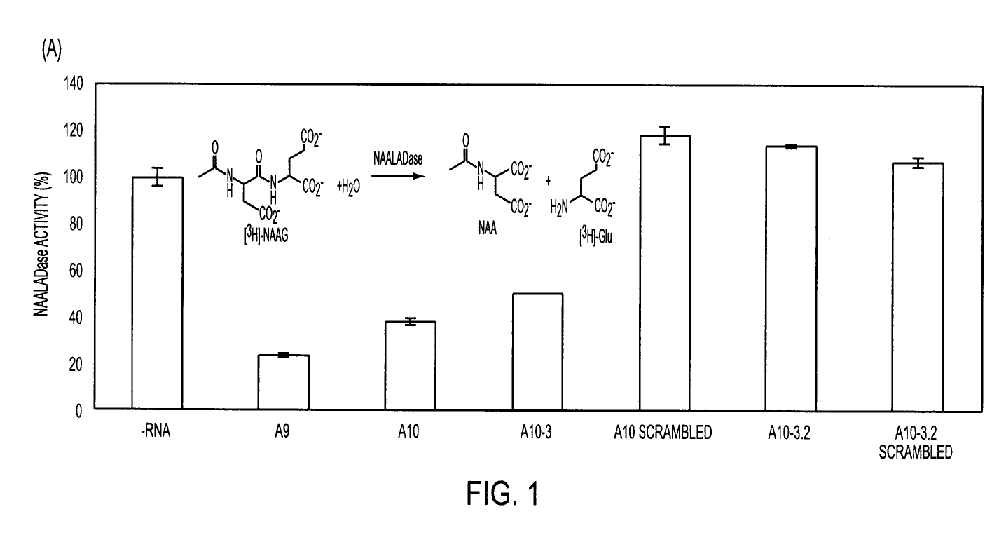

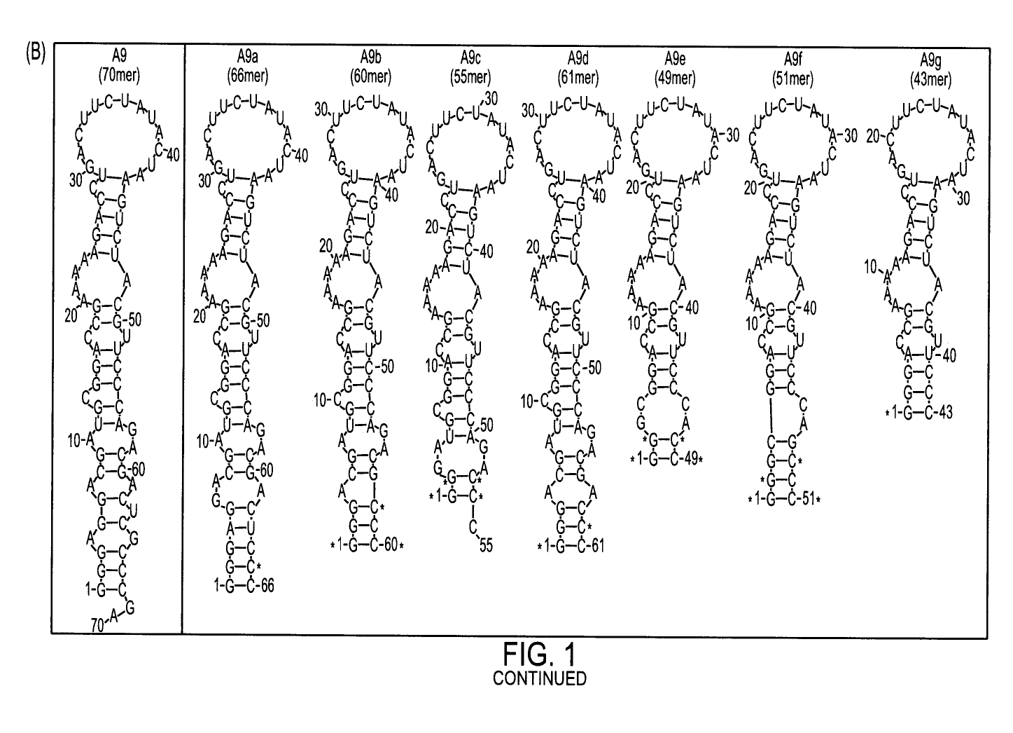

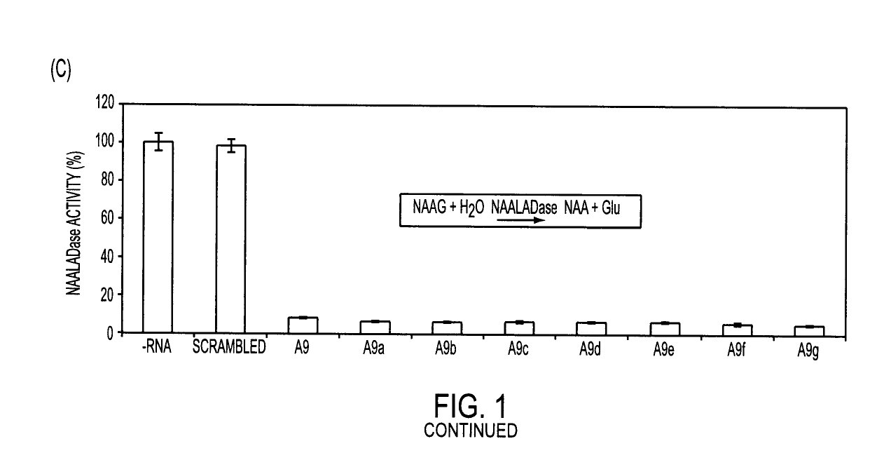

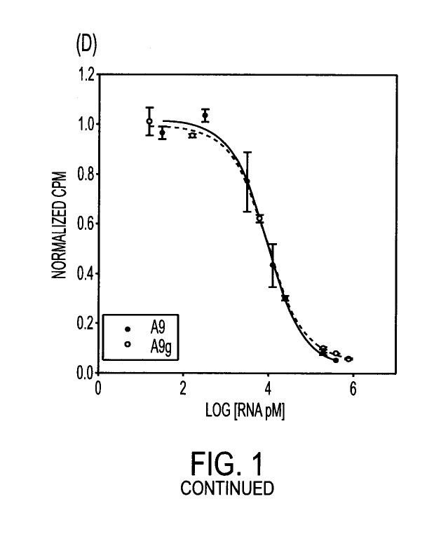

The PSMA NAALADase activity assay was modified from a previously published protocol (Xiao, Z., et al., (2000) Generation of a baculovirus recombinant prostate-specific membrane antigen and its use in the development of a novel protein biochip quantitative immunoassay. Protein Expr Purif, 19, 12-21) and performed in a final reaction volume of 200 μL. Double-distilled H₂O (ddH₂O) was used in the reaction solutions. The RNA aptamers were refolded in binding buffer (20 mM HEPES, 150 mM NaCl, 2 mM CaCl₂) at a concentration 1.667 times the final concentration desired in the activity assay (e.g., 333 nM for a final concentration of 200 nM). Refolding was accomplished by heating at 65° C. for 10 minutes, followed by cooling to 37° C. for 10 minutes. A volume of 120 μL of refolded RNA in binding buffer was added to an Eppendorf tube, was combined with 40 μL of 200 mM Tris buffer, pH 7.5, and 20 μL 10 mM CoCl₂ (final concentrations in the reaction 40 mM and 1 mM, respectively). Cobalt (II) chloride was reported to be a “stimulator of enzymatic activity” in the original NAALADase assay protocol (Xiao, Z., et al., (2000) Generation of a baculovirus recombinant prostate-specific membrane antigen and its use in the development of a novel protein biochip quantitative immunoassay. Protein Expr Purif, 19, 12-21). When this compound was omitted from the reaction, we observed increased non-specific RNA interactions. Two micrograms in 2 μL of recombinant human PSMA (4234-ZN-010) from R&D Systems (Minneapolis, Minn.) was diluted in 500 μL of 50 mM pH 7.5 Tris buffer. Ten microliters of the PSMA solution (40 ng PSMA) was added to the reaction mix, and the reaction was incubated for 5 minutes at 37° C. to promote RNA-PSMA interaction. For the experiment shown in FIG. 1A, recombinant, purified human PSMA was obtained courtesy of Dr. David Spencer (Baylor College of Medicine, Houston, Tex.). In this experiment, 2.4 μg of human recombinant PSMA protein in 10 μL of 50 mM pH 7.5 Tris buffer was added to each reaction. Ten microliters of a working solution containing 0.55 μM NAAG in H₂O having a specific activity of 10 nCi/μL of [glutamate-3,4-³H]-NAAG from Perkin Elmer (NET1082250UC) was added to the reaction mixture. The reaction was allowed to proceed for 15 minutes, mixing once by pipetting at 7.5 minutes. To halt the reaction, an equal volume (200 μL) of cold 0.1 M phosphate buffer (dibasic sodium phosphate, Na₂HPO₄) was added to the reaction mixture.

AG 1-X8 formate resin (200-400 mesh) columns from Bio-Rad (731-6221) were used to quantitate the [³H]-glutamate reaction product. Before use, the columns were equilibrated with 5 mL of ddH₂O. Half of the final reaction volume (200 μL) was added to a column. The columns were eluted twice with 2 mL of 1 M formic acid. The first elution was discarded, and the second 2 mL elution was added to 10 mL of Bio-Safe II scintillation fluid (Research Products International Corp., Mt. Prospect, Ill.). Activity was counted using a Beckman-Coulter liquid scintillation counter, and was normalized to the amount of activity obtained in the reaction with no RNA added.

Filter Binding Assays

Filter binding assays were performed as previously described (Wong, I. and Lohman, T. M. (1993) A double-filter method for nitrocellulose-filter binding: application to protein-nucleic acid interactions. Prac Natl Acad Sci USA, 90, 5428-5432). Briefly, aptamers were 5′-end labeled with ³²P using PNK. RNA was incubated for 5 minutes with various concentrations of purified, recombinant human PSMA (4234-ZN-010) obtained from R&D Systems at 37° C. The reaction mixture was spotted onto a sandwich of nitrocellulose (Protran BA 83, 0.2 μm pore size, 10 402 488, Whatman), nylon (Zeta-Probe Blotting Membranes, 162-0153, Bio-Rad), and Whatman 3MM chromatography paper (3130-6189) assembled in a dot-blot apparatus. Bound RNA was captured on the nitrocellulose filter, while unbound RNA was captured on the nylon filter. The ratio of bound:unbound RNA was calculated by exposing the filters to a storage phosphor screen and imaging with a phosphorimager.

Surface Plasmon Resonance (SPR; BIACore) Binding Measurements

Surface Plasmon Resonance (SPR) measurements were carried out using a BIACore 3000 device. 5′-biotinylated RNA was generated by transcription and gel purification as described above, except the transcription reactions were carried out in the presence of 3 mM biotin-G (Custom order from TriLink Biotechnologies, San Diego, Calif.: 5′-(Biotin) (Spacer 9) G-3′). The biotinylated RNA was immobilized on a streptavidin-coated Biacore chip (Sensor Chip SA, 13R-1003-98, General Electric Company) by injection in binding buffer at a concentration of 25 μg/mL (20 mM HEPES, pH 7.4, 150 mM NaCl, 2 mM CaCl₂). The RNA was refolded by heating to 65° C. followed by cooling to 37° C. prior to immobilization. To measure binding kinetics, fixed concentrations of purified protein were injected over the chip using the “KINJECT” function at a flow rate of 15 μL/min. After binding, the chip was regenerated by injecting 50 mM NaOH with the “QUICKINJECT” function. The binding data were fit to a 1:1 binding with mass transfer model to calculate kinetic parameters.

RNA Structural Modeling and PSMA Docking

RNA Two-Dimensional (2D) Structures Predictions.

At the 2D structural level, an RNA structure is described by the base pairs contained in the structure. The 2D structure of an RNA is predicted from the partition function, Q, defined as the sum over all the possible conformations:

$$Q = {\sum }_{s}^{ } {e}^{- Δ {G}_{S} \text{/} {k}_{B} T} ,$$

where ΔGₛ is the free energy of a given structure, s. The conformational sum

$${\sum }_{s}$$

includes all the possible secondary and pseudoknotted structures. The free energy for each given structure, ΔGₛ, is determined from ΔGₛ=ΔG_{stacks}−TΔSₗₒₒₚ where ΔG_{stacks} is the total free energy of the base stacks as determined from the Turner rules (Serra, M. J. and Turner, D. H. (1995) Predicting thermodynamic properties of RNA. Methods Enzymol, 259, 242-261) and −TΔSₗₒₒₚ is the loop free energy for the secondary and pseudoknotted structures as determined from the Vfold model (Cao, S. and Chen, S. J. (2005) Predicting RNA folding thermodynamics with a reduced chain representation model. RNA, 11, 1884-1897; Cao, S. and Chen, S. J. (2006) Free energy landscapes of RNA/RNA complexes: with applications to snRNA complexes in spliceosomes. J Mol Biol, 357, 292-312; Cao, S. and Chen, S. J. (2009) A new computational approach for mechanical folding kinetics of RNA hairpins. Biophys J, 96, 4024-4034; Cao, S., et al., (2010) Folding kinetics for the conformational switch between alternative RNA structures. J Phys Chem B, 114, 13609-13615; Chen, S. J. (2008) RNA folding: conformational statistics, folding kinetics, and ion electrostatics. Annu Rev Biophys, 37, 197-214). To predict the 2D structures, the probability Pᵢⱼ of finding nucleotides i and j to form a base pair is computed. Pᵢⱼ is calculated from the conditional partition function Qᵢⱼ: Pᵢⱼ=Qᵢⱼ/Q. Here Qᵢⱼ is the sum over all the possible conformations containing the (i,j) base pair. From the base pairing probabilities Pᵢⱼ for all the possible (i,j) pairs, we predict the 2D structures.

RNA Three-Dimensional (3D) Structures Predictions.

The 3D structures of the RNAs were generated from the predicted 2D structures (Cao, S. and Chen, S. J. (2011) Physics-Based De Novo Prediction of RNA 3D Structures. J Phys Chem B, 115, 4216-4226). The helices and loop/junctions in the structure are identified from the 2D structures. For example, the A9g structure contains two helices P1 and P2 and an internal loop L1, a bulge loop C16 and hairpin loop L2. P1 is the helix from base pair G1-C43 to base pair G7-C37 and P2 is the helix from base pair A12-U35 to base pair C15-G32. The internal loop L1 includes nucleotides from A8 to A11 and nucleotide A36. The hairpin loop includes nucleotides from G18 to A30. The 3D coordinates of the helices P1 and P2 were configured using A-form RNA helix coordinates. For the internal loop, bulge loop and hairpin loop, the fragment-based method to search for the optimal template structures from the known structures in the PDB database was employed (Cao, S. and Chen, S. J. (2011) Physics-Based De Novo Prediction of RNA 3D Structures. J Phys Chem B, 115, 4216-4226). An optimal template is defined as the template with the minimum substitution between the original loop and the template sequence. For instance, the optimal template for the internal loop L1 (5′G7AAAA3′, 5′A36C3′) was found to be the loop (5′AAAAA3′, 5′UA3′) in PDB structure 1J15A. To achieve the optimal fit of the template structure, the terminal mismatch A11-A36 was placed within the helix P2. A 3D scaffold structure was generated based on the helices and the loop template structures. In the last step, the 3D scaffold structure was further refined using AMBER energy minimization (Case, D. A., et al., (2005) The Amber biomolecular simulation programs. J Comput Chem, 26, 1668-1688).

Predicting the RNA Binding Modes on PSMA.

The binding modes of the RNA on the prostate-specific membrane antigen (PSMA) were constructed using our protein-RNA docking program. Specifically, the crystal structure of PSMA was downloaded from the Protein Data Bank (PDB code: 1Z8L) (Davis, M. I., et al., (2005) Crystal structure of prostate-specific membrane antigen, a tumor marker and peptidase. Proc Natl Acad Sci USA, 102, 5981-5986). Water, ions, and ligands were removed from the protein. The modeled RNA 3D structure was used for the RNA. Then, the putative binding modes of the RNA on PSMA were globally searched using our Fast Fourier Transform (FFT)-based macromolecular docking program MDockPP (Huang, S. Y. and Zou, X. (2010) MDockPP: A hierarchical approach for protein-protein docking and its application to CAPRI rounds 15-19. Proteins, 78, 3096-3103). MDockPP uses a hierarchical approach to construct the complexes between biological macromolecules. First, the protein was represented by a reduced model, in which each side chain on the protein surface was simplified and replaced by its center of mass. Compared with the all-atom model, the reduced model allows larger side-chain flexibility during binding mode sampling. Shape complementarity was used as a filtering criterion to generate several thousands of putative binding modes. These modes were further refined by our iteratively derived knowledge-based scoring function ITScorePP (Huang, S. Y. and Zou, X. (2008) An iterative knowledge-based scoring function for protein-protein recognition. Proteins, 72, 557-579) using the all-atom model to account for the atomic details. The top-ranked binding mode that does not interfere with the putative membrane position and the PSMA dimeric interface was selected as the predicted PSMA-RNA complex.

Cell Culture

The PSMA-positive prostate cancer cell line 22Rv1(1.7) was maintained as described in Dassie et al., 2009 in RPMI 1640 media with 10% FBS and 1% non-essential amino acids (Dassie, J. P., et al., (2009) Systemic administration of optimized aptamer-siRNA chimeras promotes regression of PSMA-expressing tumors. Nat Biotechnol, 27, 839-849). The PSMA-negative prostate cancer cell line (PC3) was maintained according to the supplier's recommendations (ATCC #CRL-1435) in DMEM/F12 media with 10% FBS. Cells were maintained at 37° C. with an atmosphere containing 5% CO₂.

Cell Binding Assay

One day prior to the binding assay, cells were plated in a 24-well plate at a density of approximately 100,000 cells per well. All subsequent procedures were performed on ice to prevent aptamer internalization. Prior to binding, each well was washed twice with 1 ml of ice-cold Dulbecco's phosphate-buffered saline in the absence of divalent cations (DPBS −/−) to remove growth media. Aptamers were 5′ end-labeled with ³²P using PNK from New England Biolabs as previously described (McNamara, J. O., et al., (2008) Multivalent 4-1BB binding aptamers costimulate CD8+ T cells and inhibit tumor growth in mice. J Clin Invest, 118, 376-386). The concentration of ³²P-radiolabeled aptamer was measured with UV-visible absorption spectroscopy, and serial dilutions ranging from 1000 nM to 30 nM were performed. To measure non-specific binding, serial dilutions were also made containing a high fixed concentration of non-radiolabeled A9g aptamer, at 10 μM. Both sets of dilutions were incubated with the cells in the 24-well plate on ice in a volume of 100 μl. After 1 hour, the binding reaction mixture was aspirated off the cells, and the cells were washed twice with 0.5 ml of ice-cold DPBS. Bound RNA was collected by washing with 0.5 ml of 0.5 N NaOH which was added to 3 ml of scintillation fluid, and activity was measured. For each dilution, specific binding was calculated by subtracting the activity of the sample with a high concentration of non-radiolabeled (“cold”) aptamer added (i.e., non-specific binding) from the sample without cold aptamer added (i.e., total binding). The data were plotted and fit to a one-site saturation binding model using the non-linear regression algorithm of the software package Sigma Plot. Experiments were performed in duplicate.

Cell Internalization Assays

22Rv1(1.7) PSMA-positive prostate cancer cells (target) and PC-3 PSMA-negative prostate cancer cells (non-target) were grown to confluency in a six-well plate. Cells were washed twice with 1 mL of DPBS prewarmed at 37° C. Cells were then blocked with 1 mL of 100 μg/mL yeast tRNA prewarmed at 37° C. After 15 min the block was removed and 100 pmol RNA aptamer in DPBS was added to cells for 30 min at 37° C. with 5% CO₂. Cells were washed once with ice-cold DPBS followed by two washes of ice-cold 0.5M NaCl in DPBS. The internalized RNA was recovered using TRIzol reagent. Quantitative RT-PCR was performed using the iScript One-Step RT-PCR Kit with SYBR Green (Cat#170-8893) from Bio-Rad Laboratories (Hercules, Calif.). Samples were normalized to an internal RNA reference control. Specifically, 0.5 pmol/sample m12-23 aptamer (McNamara, J. O., et al., (2008) Multivalent 4-1 BB binding aptamers costimulate CD8+ T cells and inhibit tumor growth in mice. J Clin Invest, 118, 376-386) was added to each sample along with TRIzol as a reference control. Primer sets included the internal reference primer set for m12-23 (Sel1), the A9g primer set (amplifies A9, A9g, and A9g.6), the A10 primer set (amplifies A10 and A10-3.2), and the A10-3.2 scrambled primer set. Samples were first normalized to the internal reference RNA (m12-23) and then according to the relative amount of RNA internalized versus the non-target control cells (PC3).

Primer sequences for the quantitative RT-PCR are as follows: Sell 5′ primer: 5′-GGGGGAATTCTAATACGACTCACTATAGG GAGAGAGGAAGAGGGATGGG-3′; Sel1 3′ primer 5′-GGGGGGATCCAGTACTATCGACCTCT GGGTTATG-3′; A9g 5′ primer: 5′-TAATACGACTCACTATAGGGACCGAAAAAGACC-3′; A9g 3′ primer: 5′-GGGAACGTAGACTTAG-3′; A10 5′ primer: 5′-TAATACGACTCACTATAGGGAGGA CGATGCGG-3′; A10-3.2 3′ primer: 5′-AGGAGTGACGTAAACATG-3′; A10-3.2 scrambled 5′ primer: 5′-TAATACGACTCACTATAGGGGCATGCCTAGCT-3′; A10-3.2 scrambled 3′ primer: 5′-CCGCGCATAAGCCATGGG-3′.

Results

Rational Truncation of A9 PSMA RNA Aptamer

The PSMA RNA aptamers A9 and A10 have been selected for their ability to inhibit PSMA's enzymatic activity (Lupold, S. E., et al., (2002) Identification and characterization of nuclease-stabilized RNA molecules that bind human prostate cancer cells via the prostate-specific membrane antigen. Cancer Res, 62, 4029-4033). Because PSMA's enzymatic activity has been implicated in carcinogenesis (metastatic potential) (Lapidus, R. G., et al., (2000) Prostate-specific membrane antigen (PSMA) enzyme activity is elevated in prostate cancer cells. Prostate, 45, 350-354), optimized, truncated versions of these inhibitors promise to be valuable agents not only for targeted imaging and therapy of prostate cancer but also to directly inhibit PSMA's pro-metastatic functions. We used the NAALADase assay to assess the inhibitory activity of previously described, truncated versions of the A10 RNA aptamer: A10-3 (56 mer) and A10-3.2 (39 mer) (FIG. 1A). The NAALADase activity of PSMA hydrolyzes N-acetylaspartylglutamate (NAAG) to N-acetylaspartate and glutamate (FIG. 1A; insert). As previously described, A10-3 retains NAALADase inhibitory activity, albeit less efficiently compared to the full-length A10 and A9 RNA aptamers. In contrast, A10-3.2 (39 mer) had no NAALADase inhibitory activity. This was confirmed at higher RNA concentrations up to 3.8 μM (data not shown). Scrambled versions of the A10 and A10-3.2 aptamers were used as negative controls in this assay. These scrambled aptamers have the same number of nucleotides and base composition as their wild-type counterparts but possess a “scrambled” sequence.

As previously described, A9 is a better inhibitor of PSMA enzymatic activity compared to A10. Thus, we set out to determine the NAALADase inhibitory activity of various truncations of the A9 aptamer. Previous attempts at truncating the A9 aptamer have proved unsuccessful. Thus, rather than performing a series of base deletions from the 3′ end, we reasoned that maintaining the overall structure of the PSMA-interacting region of the aptamer would be essential for retaining activity. To this end, a series of 5′ and 3′-end base deletions were made, and the RNA secondary-structure prediction program RNAStructure 4.6 was used to select those truncations which retained the predicted secondary structural motifs of the full-length A9 aptamer (FIG. 1B). In addition, selective base changes were made at the 5′ and 3′ ends to maintain a T7 transcription start-site (5′GGG) and maintain base-paring complementarity at the 3′ end.

Seven initial truncated versions of the A9 aptamer were designed (A9a through A9g) with lengths ranging from 66 bases (A9a) to 43 bases (A9g). The NAALADase assay was used to assess inhibition of PSMA enzymatic activity by the various truncations. A scrambled RNA aptamer sequence (71 mer) did not inhibit enzymatic activity. Remarkably, all seven truncations inhibited PSMA NAALADase activity as well as full-length A9 under these assay conditions (800 nM RNA) (FIG. 1C). We next determined the inhibitory potency of the shortest truncation, A9g (43 mer) compared to the full-length A9 aptamer. Inhibition was tested over a range of RNA concentrations (20 pM to 800 nM). Both A9g (43 mer) and A9 (70 mer) inhibited NAALADase activity with an IC₅₀ of 10 nM under the assay conditions (FIG. 1D), suggesting that A9g, like A9, retains key structural/sequence elements important for inhibition of PSMA enzymatic activity.

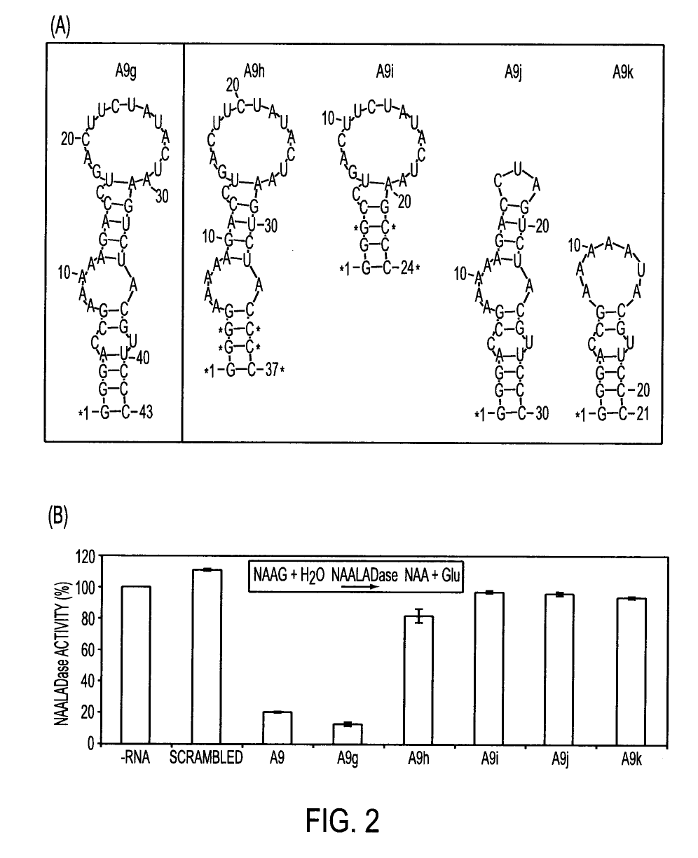

A second series of truncations were made in an attempt to further decrease the length of the A9g aptamer and to assess structural and sequence elements important for PSMA inhibition (FIG. 2A). The truncations A9h (37 mer) and A9i (24 mer) retain sequence and structural loop elements of A9g, while A9j (30 mer) and A9k (21 mer) retain sequence and structural stem elements of A9g (FIG. 2A). Interestingly, unlike A9 and A9g, none of these additional truncations (A9h-A9k) exhibited inhibitory activity under the assay conditions (200 nM RNA concentration) (FIG. 2B). Together, these results suggest that key sequence and/or structural elements for PSMA inhibition are present within bases 1-43 of the A9g aptamer.

A9g Binds to PSMA with High Affinity and Specificity

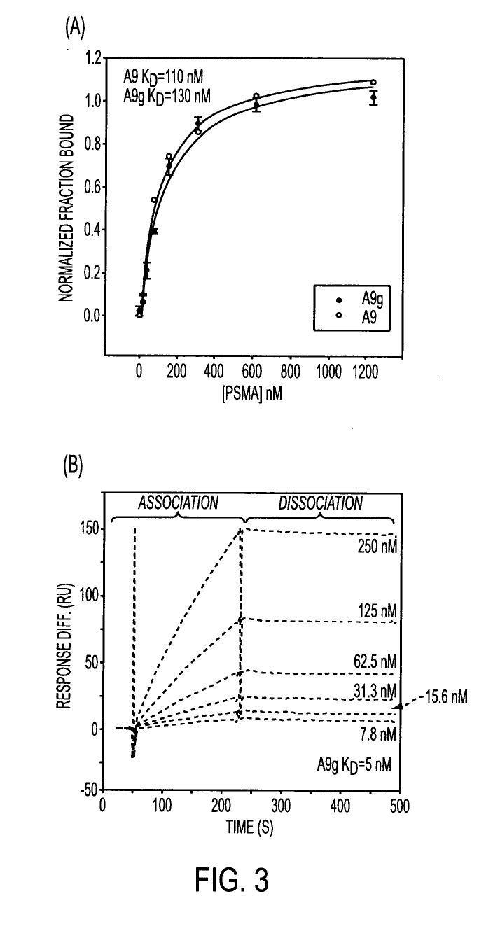

The NAALADase activity assay provides an indirect measurement of the interaction of the PSMA aptamers with PSMA. To determine the binding profile of the A9g aptamer for PSMA we performed filter-binding assays (FIG. 3A) and Surface Plasmon Resonance (SPR/BIACore) with recombinant, purified human PSMA protein (FIG. 3B). As determined by the filter binding assay, the A9g aptamer retains the same binding profile as the full-length A9 (FIG. 3A). A more extensive measure of binding by analyzing kinetic interaction data using SPR/BIACore was also performed. In these experiments, biotinylated A9g RNA was immobilized on streptavidin-coated gold chips. A solution containing the analyte of interest (recombinant purified PSMA protein) was injected over the chip during an association phase, allowing for measurement of the binding on-rate (kₒₙ). After the injection was halted, the rate of dissociation (k_{off}) was measured. By repeating these measurements at various analyte (PSMA) concentrations, an accurate estimation of binding was determined (K_{D}=k_{off}/Kₒₙ). The K_{D} of A9g for PSMA ranged from 5 nM to 30 nM in triplicate experiments (lowest value shown) (FIG. 3B).

Structure-Function Analysis of A9g Binding to PSMA

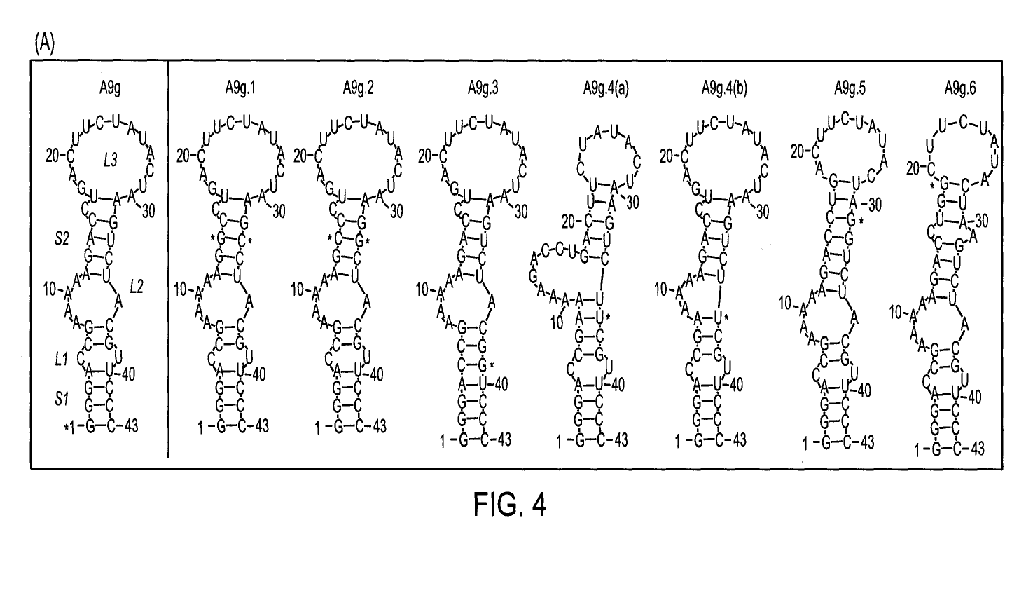

A series of base changes were introduced within A9g in an attempt to identify the sequence/structural elements necessary for binding to PSMA. Inherent in these experiments is the assumption that the base changes only create local changes in the RNA structure and not a global change in folding. For these experiments, the A9g aptamer was divided into two stem regions (S1 and S2) and three loop regions (L1, L2 and L3) (FIG. 4A). Base changes were made to either preserve or disrupt these various structural elements. The RNA-secondary structure prediction algorithm, RNAStructure 4.6, was used to predict folding of the modified A9g RNAs (A9g.1-A9g.6).

To address the importance of the S2 stem sequence, the A-U base pair in the stem region S2 was replaced with either a G-C or a C-G base pair (A9g.1 and A9g.2 respectively) (FIG. 4A). A9g.1 and A9g.2 were predicted to retain the overall secondary structure as A9g (FIG. 4A). As predicted, A9g.1 and A9g.2 resulted in RNA aptamers with comparable inhibitory activity as A9g (FIG. 4B). In contrast, a base change within S2 which was predicted to lengthen the stem (A9g.5) resulted in loss of PSMA inhibitory activity suggesting that the overall structural and not sequence elements of S2 are important for the RNA's inhibitory function. We next addressed the importance of each loop (L1, L2, and L3) by introducing base changes that would disrupt the predicted folding of the loops (A9g.3, A9g.4, and A9g.6 respectively). With the exception of A9g.4, all base changes completely abrogated the ability of the RNA aptamers to inhibit PSMA enzymatic activity (FIG. 4B) suggesting that the loops are required for function. In the case of A9g.4, inhibitory activity was decreased by approximately 50% compared to A9g. Interestingly, two distinct secondary structures (A9g.4a and A9g.4b) with similar minimum free energies (ΔGs) were predicted for A9g.4 (FIG. 4A). The predicted free energies of these two structures were −9.9 kcal·mol⁻¹ and −9.4 kcal·mol⁻¹, respectively. To assess whether loss of inhibitory function correlates with loss of binding to PSMA we performed filter binding assays to determine binding of A9g.3-A9g.6 to recombinant PSMA (FIG. 4C). With the exception of A9g.4, the binding capacity (Bₘₐₓ) of PSMA for these mutants was severely diminished. The binding of A9g.4 mirrored its inhibitory activity (FIG. 4B), with a binding capacity for PSMA of approximately 50% compared to A9g.

Assessment of Binding Specificity of A9g to PSMA

Binding specificity of the A9g aptamer for PSMA was determined using SPR/BIACore (FIG. 4D; left panel). Binding specificity was assessed by comparing the kₒₙ, and k_{off} rates of A9g for recombinant PSMA protein (target) to the Kₒₙ, and k_{off} rates of A9g for non-target proteins (BSA and HER2). For these experiments, biotinylated A9g RNA was immobilized on streptavidin-coated gold chips. No appreciable interaction between A9g and the non-target proteins (BSA and HER2) was measured (FIG. 4D; left panel). Lack of binding of A9g.6 to PSMA was also confirmed with SPR/BIACore (FIG. 4D; right panel). In addition, there was no measurable binding of A9g.6 to the non-target proteins (BSA and HER2). These data provide confirmation of binding specificity of A9g for PSMA (FIG. 4D).

RNA Tertiary Structure Predictions and RNA Protein Docking Studies

With the exceptions of A9g.1 and A9g.2 which were designed to have the same secondary structure as wild-type A9g, all of the other A9g-derivatives experienced a significant decrease in their ability to inhibit and bind PSMA. It may be that each of the predicted secondary structural elements examined play a role in the aptamer's binding to PSMA. Alternatively, any of changes made to the predicted structural elements may disrupt the “global” folding of the RNA, rendering it inactive.

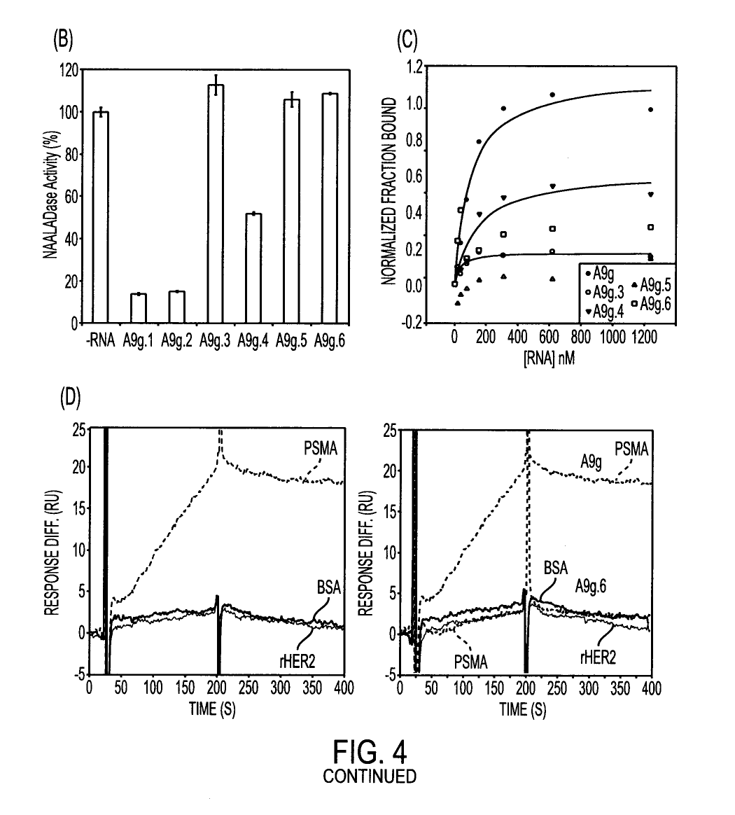

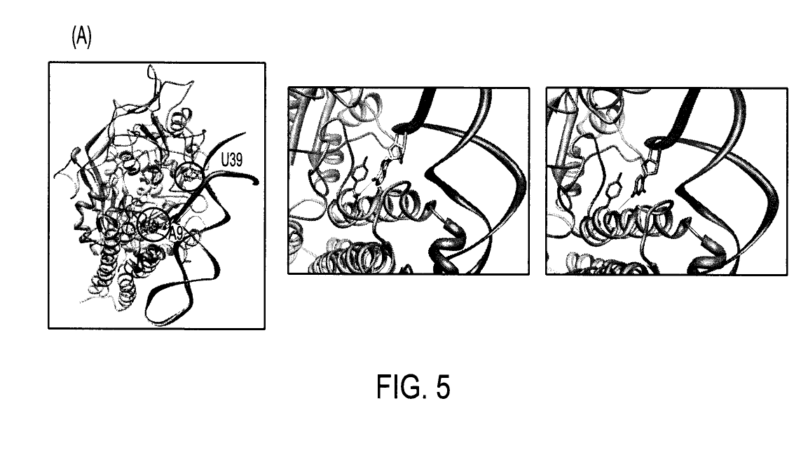

To provide additional insight into the interaction of the A9g RNA aptamer with PSMA, a tertiary structure model of A9g was created. The predicted tertiary structure of A9g was computationally docked to a crystal structure of PSMA (Davis, M. I., et al., (2005) Crystal structure of prostate-specific membrane antigen, a tumor marker and peptidase. Proc Natl Acad Sci USA, 102, 5981-5986) (FIG. 5A; left panel). Interestingly, the RNA-protein docking analysis revealed two bases, adenosine at position 9 (A9) and uridine at position 39 (U39), that were predicted to interact directly with PSMA. The amine group of A9 forms a hydrogen bond with a backbone carbonyl of PSMA, and U39 forms multiple close van der Waals interactions with PSMA side-chains. On the basis of these predictions, base changes were made to retain the hydrogen bond at position A9 (FIG. 5A; compare middle and right panels) and to test the necessity of U at position 39. Specifically, the uridine at position 39 was replaced with either an adenosine (A9g.7; U39A) or a cytosine (A9g.8; U39C) and the adenosine at position 9 with a cytosine (A9g.9; A9C) (FIG. 5A; right panel). Predicted secondary structures for these A9g variants are shown in FIG. 5B. Not surprisingly, the A9g (A9C) variant retained PSMA inhibitory activity, albeit less effectively compared to A9g (FIG. 5B). In contrast, the A9g (U39A), A9g (U39C) and A9g (U39G) variants completely lost inhibitory activity (FIG. 5B). Notably, unlike the A9g (U39G) variant (identical to A9g.3, FIG. 4A), the A9g (U39A) and A9g (U39C) variants were not predicted to alter the secondary structure of A9g (FIG. 5B). These data suggest that sequence conservation (uridine) at position 39 may be more important than the overall structure of the L1 loop for conferring the RNA aptamer's inhibitory function.

Based on the above data, we hypothesized that a further truncation of A9g which retains uridine at position 39 should result in an RNA aptamer with comparable PSMA inhibitory activity to A9g. To test this hypothesis, we removed the most distal G-C base-pair of A9g (A9L; 41 mer). We also introduced a base change at the first position to maintain the 5′-GGG T7 RNA polymerase transcription start (FIG. 5C; left panel). As predicted, A9L was equally as effective as A9g at inhibiting PSMA enzymatic activity (FIG. 5C; right panel). Elimination of additional bases from the 5′ or 3′ termini (e.g. A9h; 37 mer) abrogated inhibition of PSMA enzymatic activity (FIG. 5C; right panel). These findings were consistent with altered folding of these shorter RNAs as predicted using the RNA secondary structure prediction algorithm (RNAStructure 4.6) and loss of sequence elements (e.g. U at position 39) required for function.

A9g and A9L Bind to and Internalize into PSMA Positive Prostate Cancer Cells.

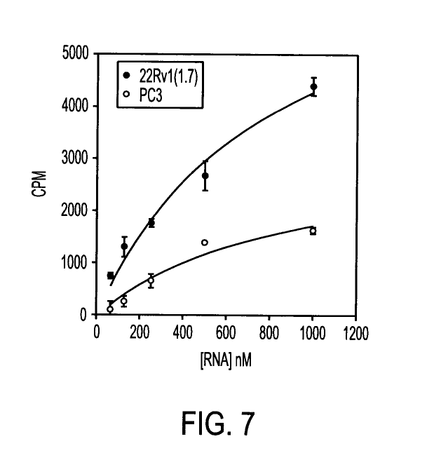

Binding of A9g to PSMA expressed on the surface of prostate cancer cells was confirmed by incubating varying amounts of ³²P-labeled A9g with either PSMA-positive (22Rv1 clone 1.7) or PSMA-negative (PC-3) prostate cancer cells on ice (to prevent internalization into the cells) (FIG. 7). The PSMA-expressing cells were found to have an approximately two-fold higher binding capacity for A9g compared to the PSMA-negative cells (FIG. 7).

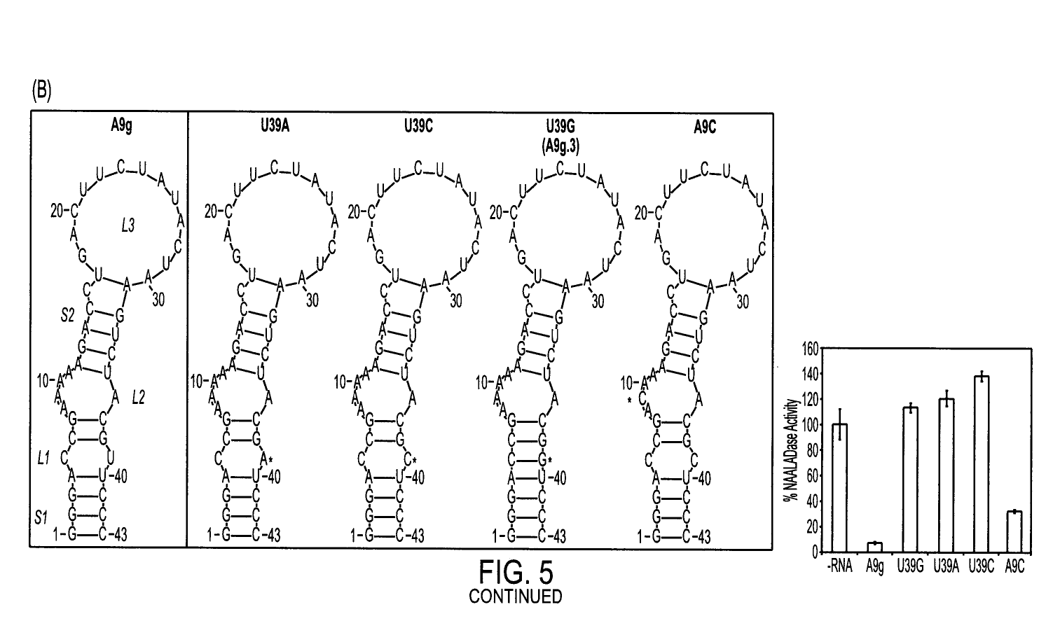

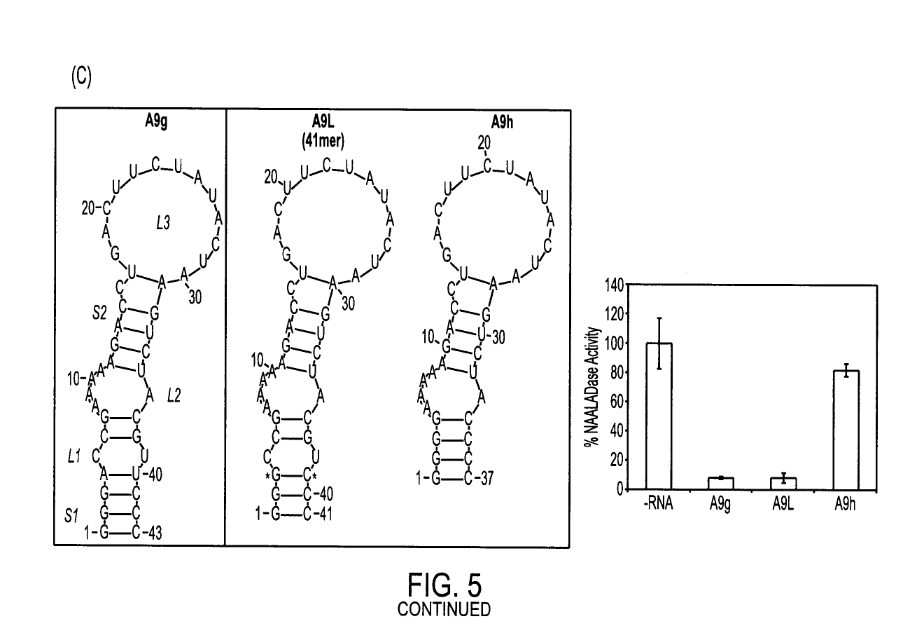

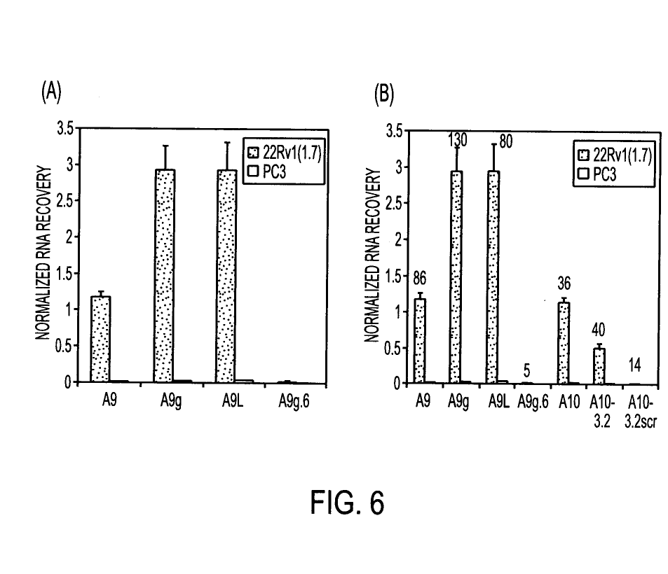

Aptamers that bind to cell-surface proteins (e.g. cancer epitopes) can be developed for imaging applications. In addition, aptamers with cell-internalizing properties can be harnessed for delivery of therapeutic agents into target cells. The A9 and A10 RNA aptamers were both demonstrated to be effective at delivering cargos which require internalization, such as cytotoxic drugs and siRNAs. For therapeutic development, the A10 aptamer was further truncated to 39 bases (A10-3.2) while retaining the ability to bind to PSMA on the surface of cells and deliver its therapeutic siRNA cargo into PSMA-expressing prostate cancer cells. Unfortunately, the shorter A10-3.2 aptamer no longer exhibits PSMA inhibitory activity (FIG. 1A). Because inhibitory activity, binding and internalization ability do not necessarily coincide, we performed an internalization assay to assess whether the shorter A9 aptamer variants (A9g and A9L), which retain PSMA inhibitory activity (FIG. 5C), internalize into PSMA-expressing prostate cancer cells (FIG. 6A). Full-length A9, A9g (43 mer) and A9L (41mer) aptamers were incubated with either PSMA-positive (22Rv1 clone 1.7) or PSMA-negative (PC-3) prostate cancer cells at 37° C. to enable cell internalization. Cells were washed with a high salt wash buffer containing 0.5 M NaCl to remove non binders or aptamers bound to the surface of the cells. Internalized aptamers were recovered by Trizol extraction. The efficiency of internalization for each RNA aptamer was assessed using quantitative RT-PCR (FIGS. 6A-6B). No loss in internalization ability was observed for the truncated A9 variants (A9g and A9L) compared to the full-length A9 RNA aptamer (FIG. 6A). As expected, A9g and A9L retained specificity for cells expressing PSMA (FIG. 6A). Importantly, A9g and A9L internalized more efficiently into PSMA expressing prostate cancer cells compared to A10 and the A10 truncated variants (A10-3 and A10-3.2) (FIG. 6B). No internalization was observed with a scrambled A10-3.2 aptamer sequence or with a functionally inactive mutant of A9g (A9g.6) (FIGS. 6A-6B). All A10 and A9 RNA aptamer derivatives retained specificity for PSMA expressing cells (22Rv1 clone 1.7) over PSMA-negative cells (PC-3) (FIG. 6B). The fold increase of RNA recovered from PSMA-expressing cells vs. RNA recovered from PC-3 cells is shown for each RNA aptamer. No statistically difference in internalization is observed for A10 and A10-3.2 (p=0.1). In contrast, the truncated A9 variants (A9g and A9L) internalized more efficiently into PSMA-expressing cells compared to either the full-length A9 aptamer (p<0.1) or A10 aptamers. Together these data confirm that the truncated A9 aptamer variants (A9g and A9L) retain target-specific cell-internalizing properties and can thus be developed into effective targeted delivery agents for prostate cancer.

Discussion

Here the inventors describe a “rational truncation” approach that takes advantage of computer-generated RNA structure models to facilitate the truncation of RNA aptamer sequences post-selection. This approach enabled the inventors to engineer truncated versions of the PSMA A9 aptamer that retain binding affinity, specificity and functionality. Computer-generated RNA secondary structure models were used to remove bases from both the 5′- and 3′-termini of the RNA and introduce base changes to conserve those secondary structural elements that are predicted to be necessary for binding to PSMA. This analysis resulted in a 27-base truncation of the PSMA A9 RNA aptamer, yielding an RNA oligonucleotide of 43 nucleotides long (A9g), which binds to recombinant PSMA with nanomolar affinity (K_{D}=5 nM) (FIG. 3B) and retains PSMA inhibitory activity (FIG. 1D). Importantly, we show that like A9, A9g retains the ability to internalize into PSMA-expressing prostate cancer cells (FIGS. 6A-6B) and thus could be used for targeted delivery of therapeutic agents (toxins, siRNAs, and radionuclides). In addition to computer-generated RNA secondary structure models, we combined predictive RNA tertiary structure models with protein docking studies to obtain further insights into the A9g-PSMA interaction (FIGS. 5A-5C). This analysis revealed key nucleotides within A9g critical for binding to PSMA (FIG. 5A). Furthermore, this analysis enabled us to perform an additional 2-nucleotide truncation of A9g resulting in a 41 nucleotide long RNA oligonucleotide (A9L) with comparable binding affinity and activity to A9 and A9g (FIG. 5C).

The successful truncation of the A9 PSMA aptamer is of importance in light of recent data directly implicating PSMA's enzymatic activity in promoting carcinogenesis. PSMA has multiple catalytic activities, including NAALADase, folate carboxypeptidase, and dipeptidyl peptidase IV activity. Recent studies have suggested a role for PSMA enzymatic activity in cell migration and activation of oncogenic pathways. Importantly, inhibition of PSMA enzymatic activity by small molecule inhibitors abrogates PSMA-mediated carcinogenesis. Here the inventors have shown that the A9g (43 mer) and A9L (41 mer) aptamers, like A9, retain the ability to inhibit PSMA's NAALADase activity (FIG. 5C) and thus could be employed as therapeutic inhibitors of PSMA. In contrast, a previously described truncated version of the A10 PSMA aptamer (A10-3.2; 39 mer), which retains binding to PSMA is unable to inhibit PSMA NAALADase activity (FIG. 1A).

The A10-3.2 aptamer has been successfully used by us to deliver siRNAs targeting cancer prosurvival genes to PSMA-expressing prostate cancer cells. In this context, the truncated aptamer serves solely as a delivery tool for the therapeutic siRNA cargo. In principle, conjugation of therapeutic siRNAs to the A9g and A9L aptamers, which we demonstrate internalize efficiently and specifically into PSMA-expressing cells (FIGS. 6A-6B), could result in dual function targeted reagents capable of inhibiting multiple carcinogenic pathways (PSMA and prosurvival genes). An aptamer-siRNA conjugate with dual function has been previously described for the treatment of HIV infected cells (Zhou, J., et al., (2008) Novel dual inhibitory function aptamer-siRNA delivery system for HIV-1 therapy. Mol Ther, 16, 1481-1489). In this report, an inhibitory aptamer against gp120 was tethered to a siRNA against tat/rev, two viral genes which drive replication of the virus. The aptamer-siRNA combination reduced HIV infectivity and replication in cultured T cells and suppressed HIV-1 viral loads reversing CD4+ T cell decline in a humanized mouse model of HIV (Neff, C. P., et al., (2011) An aptamer-siRNA chimera suppresses HIV-1 viral loads and protects from helper CD4(+) T cell decline in humanized mice. Sci Transl Med, 3, 66ra66).

The information provided by the theoretical secondary and tertiary RNA structure models is used not only to guide in the truncation of long RNA oligonucleotide sequences (as described herein) but also to enable the modification of key nucleotides in order to improve overall aptamer quality. While large-scale, high-quality, cGMP-grade synthesis of long RNA oligonucleotide aptamers (60-100 nucleotides long) remains a rate limiting step to their therapeutic potential, other in vivo properties of these RNAs, such as their pharmacokinetics (PK) and pharmacodynamics (PD), can also hinder their therapeutic utility (reviewed in Keefe, A. D., et al., (2010) Aptamers as therapeutics. Nat Rev Drug Discov, 9, 537-550). Several ways to optimize the PK/PD of aptamers have been described. These include (1) the use of modified nucleotides that impart nuclease resistance resulting in RNA aptamers with longer half-lives in the blood and (2) chemical conjugation of high-molecular weight molecules (e.g. 20-40 kDa PEG) to prevent exclusion by renal filtration. While 2′-fluoro modified pyrimidines are usually incorporated into RNA aptamers during the selection process, additional modifications are introduced post selection, using a trial-and-error approach that is laborious and is not guaranteed to work for all aptamers. In principle, theoretical RNA structure algorithms like the ones described herein, can be utilized to identify bases that when modified (with synthetic bases) may increase the overall thermodynamic stability and nuclease resistance of these RNA aptamers without loss of function. Likewise, these algorithms can be used to identify critical residues that cannot tolerate modifications (FIG. 5A).

In conclusion, our studies highlight the utility of theoretical RNA secondary and tertiary structure models and protein docking studies for guiding the truncation of RNA aptamers in order to enable and expedite large-scale chemical synthesis of these RNAs for clinical applications. Importantly, these efforts have resulted in a truncated PSMA A9 aptamer that due to its shorter sequence length is now amenable to large-scale chemical synthesis for targeted therapeutic applications in the setting of prostate cancer. Finally, the ability to directly test the computer-generated structural predictions using robust functional assays (binding and enzymatic activity) can enable the refinement of current RNA prediction algorithms. Once refined, these theoretical models can be applied to optimize other aptamers with therapeutic potential.

### Example 2

### Effect of A9g on Reducing Motility and Invasion of PSMA+ Prostate Cancer Cells in Culture and In Vivo

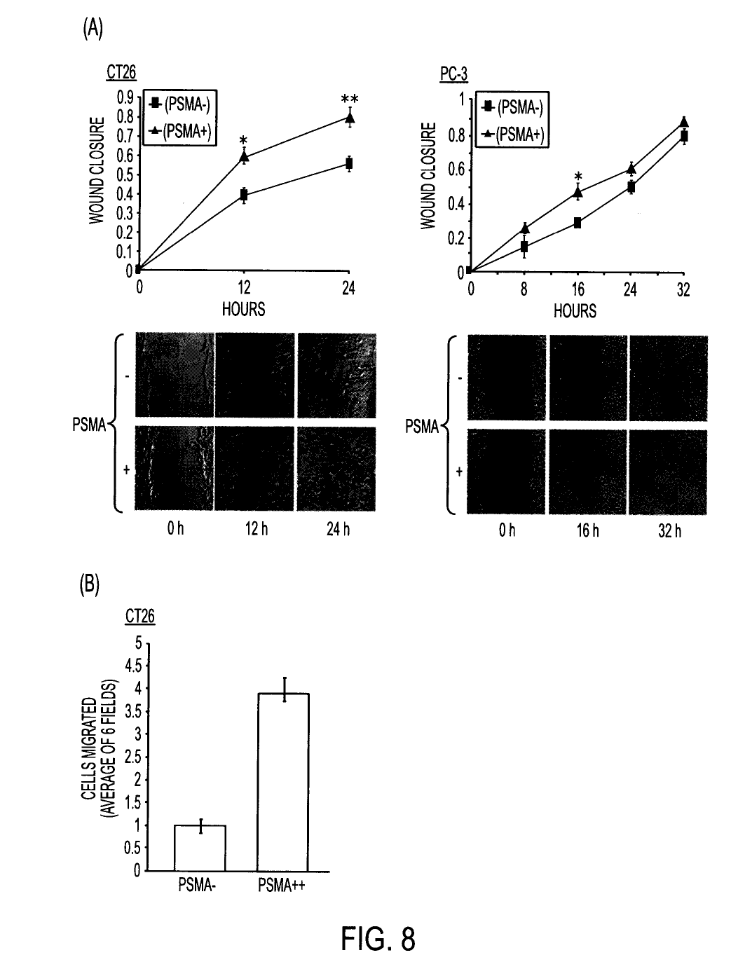

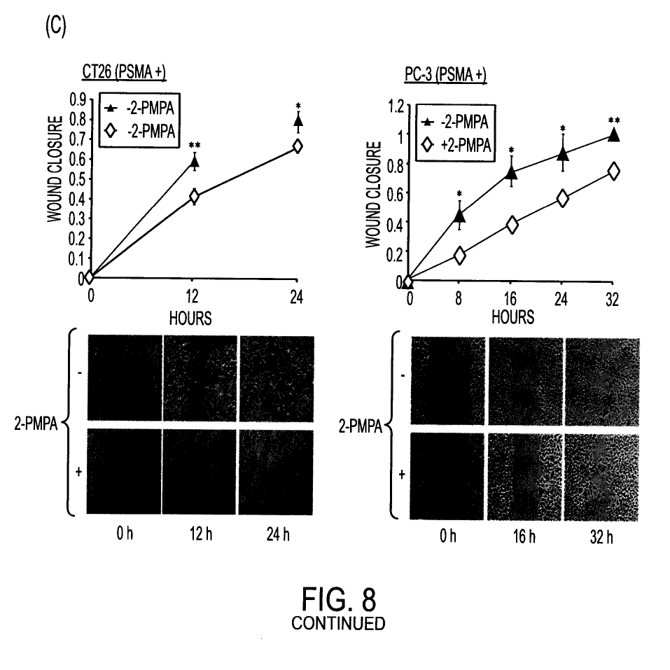

Experiments were performed to evaluate the effect of the A9g aptamer on reducing motility and invasion of PSMA and prostate cancer cells in culture and in vivo. It was observed that PSMA expression promotes cell migration (FIGS. 8A-8C).

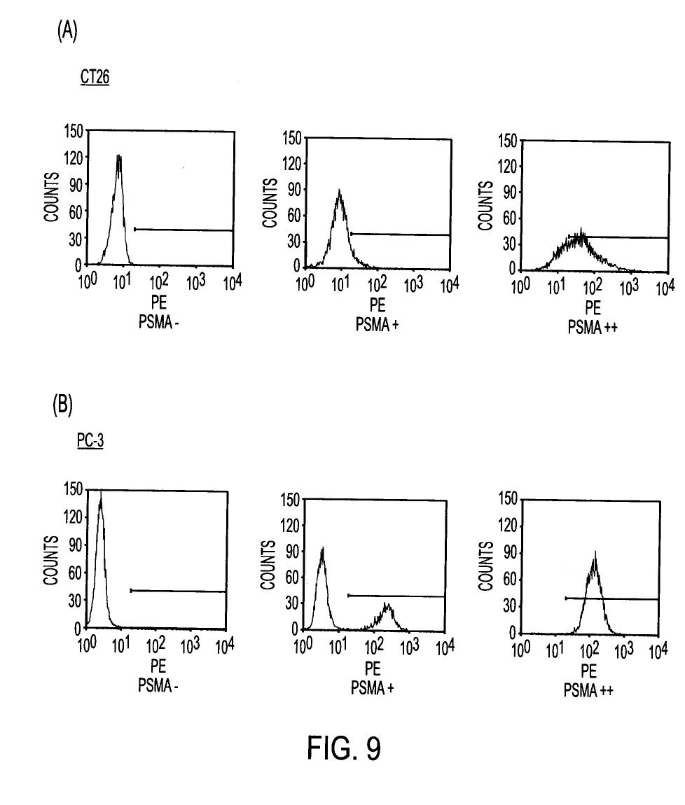

Experiments were also performed to evaluate the expression of PSMA in certain cancer cell lines (FIGS. 9A-9B). These data show that we were able to select for cell lines with high heterogenous human PSMA expression. The PC-3 cell line is of human origin (prostate cancer) while the CT26 cell line is of mouse origin (colorectal carcinoma).

Experiments were performed to evaluate PSMA expression and proliferation (FIG. 10). The results from these experiments show that PSMA expression in cells does not promote cellular proliferation.

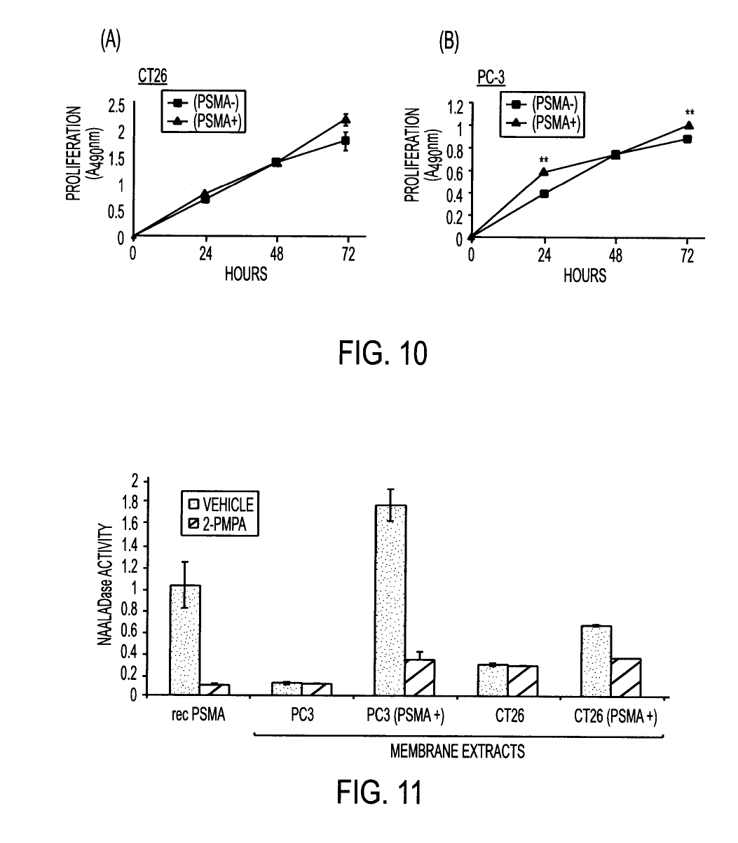

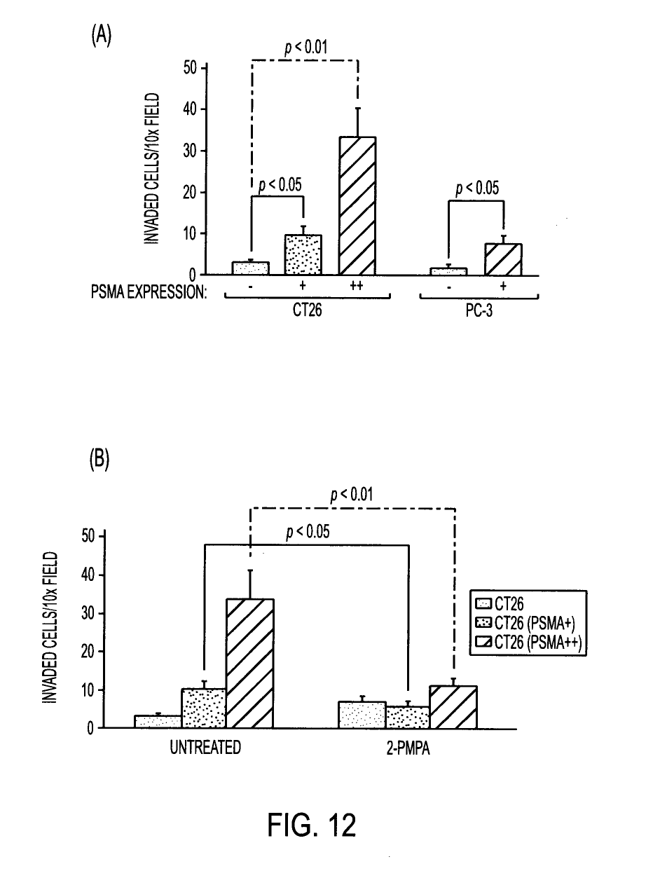

Experiments were performed to evaluate the inhibition of PSMA enzymatic activity on cell membrane extracts (FIG. 11) These experiments were performed to evaluate the activity of exogenously expressed PSMA in CT26 and PC3 cell lines. The experiments show that exogenously expressed PSMA is active, capable of producing NAALADase activity. Furthermore, PSMA enzymatic activity can be inhibited using 2-PMPA a small molecule inhibitor of PSMA. Experiments were performed to evaluate PSMA expression, and whether it promotes cell invasion (FIG. 12A-12B). These experiments demonstrate that PSMA expression promotes cell invasion. Importantly, cells invasion positively correlates with PSMA expression levels and can be inhibited by 2-PMPA.

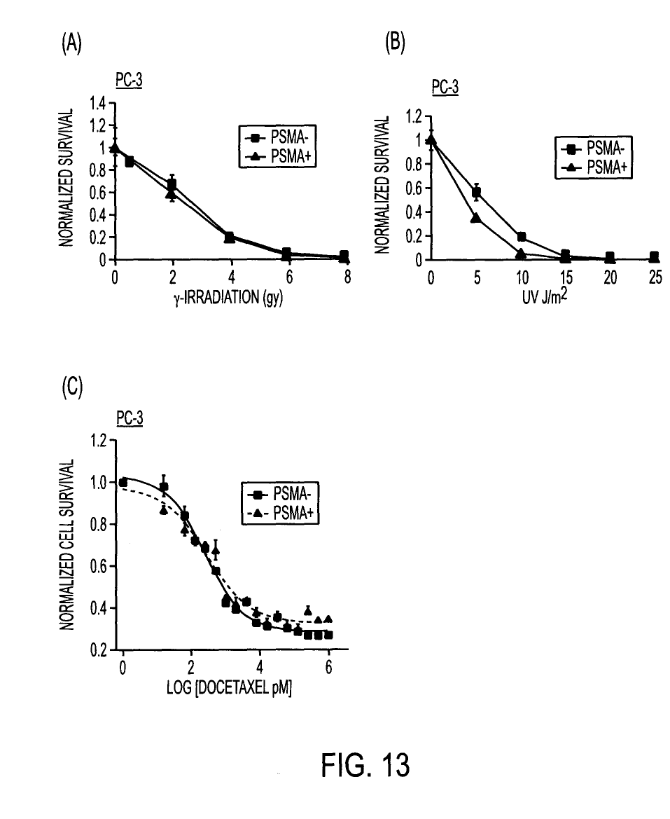

Experiments were performed to evaluate whether PSMA expression has an effect on cell survival (FIGS. 13A-13C). It was observed that PSMA expression does not affect cell survival.

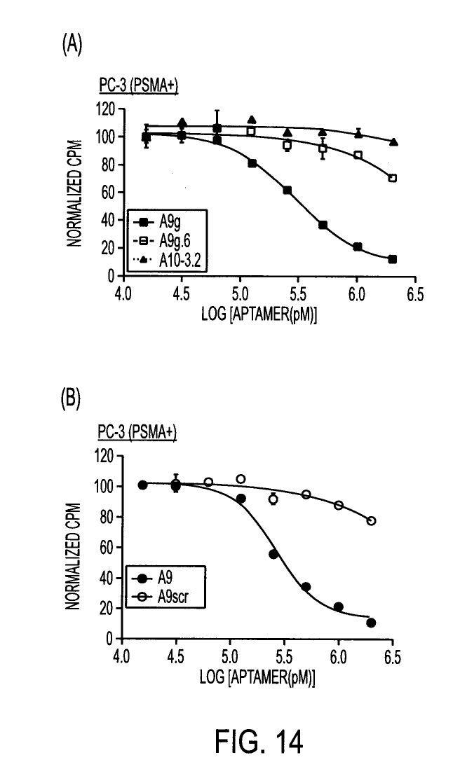

Experiments were performed to evaluate the inhibition of cell-derived PSMA enzymatic activity by synthetic RNA aptamer ligands (FIGS. 14A-14B). These data reveal that Aptamer A9g is a potent inhibitor of PSMA enzymatic activity (NAALADase activity) whereas, a mutant aptamer (A9g.6) that does not bind to PSMA does not inhibit PSMA enzymatic activity. Importantly, a different PSMA aptamer (A10-3.2) which retains binding to PSMA, does not inhibit its enzymatic activity.

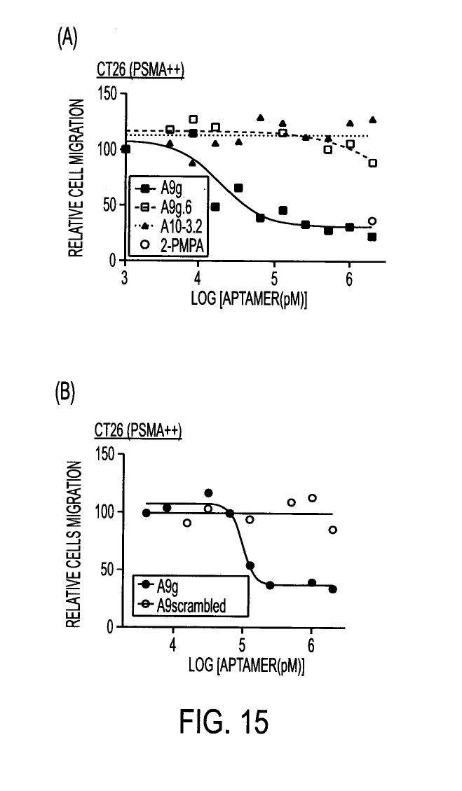

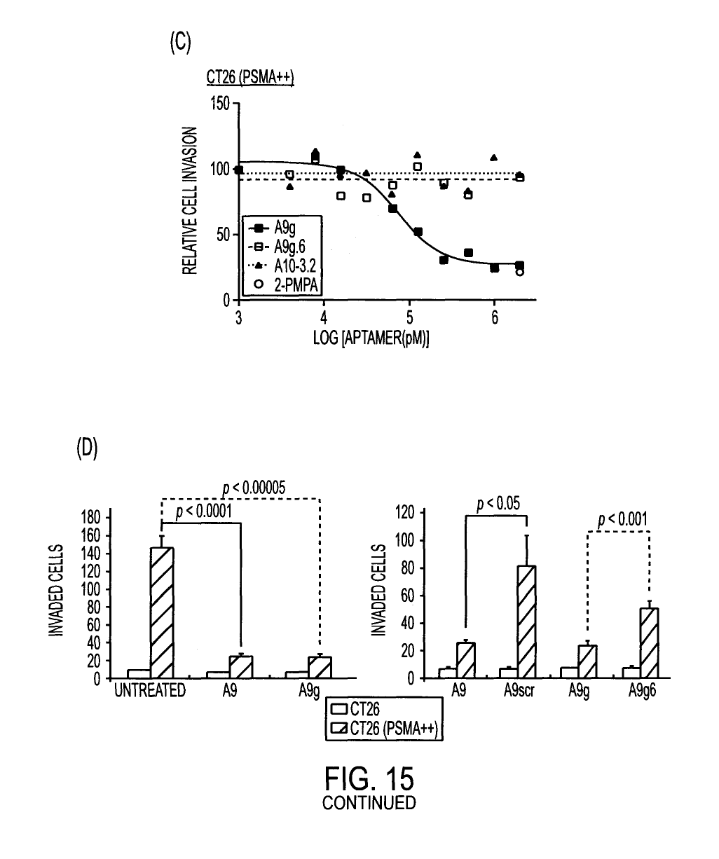

Experiments were performed to evaluate the inhibition of PSMA-mediated carcinogenesis by synthetic RNA aptamer ligand (FIGS. 15A-15D). These data reveal that aptamer A9g is a potent inhibitor of PSMA-mediated cell migration/invasion. The full length aptamer (A9) is also a potent inhibitor of cell migration/invasion. In contrast, aptamer A9g.6 (non-binder) and aptamer A 10-3.2 (inert binder) do not affect PSMA-mediate cell migration/invasion.

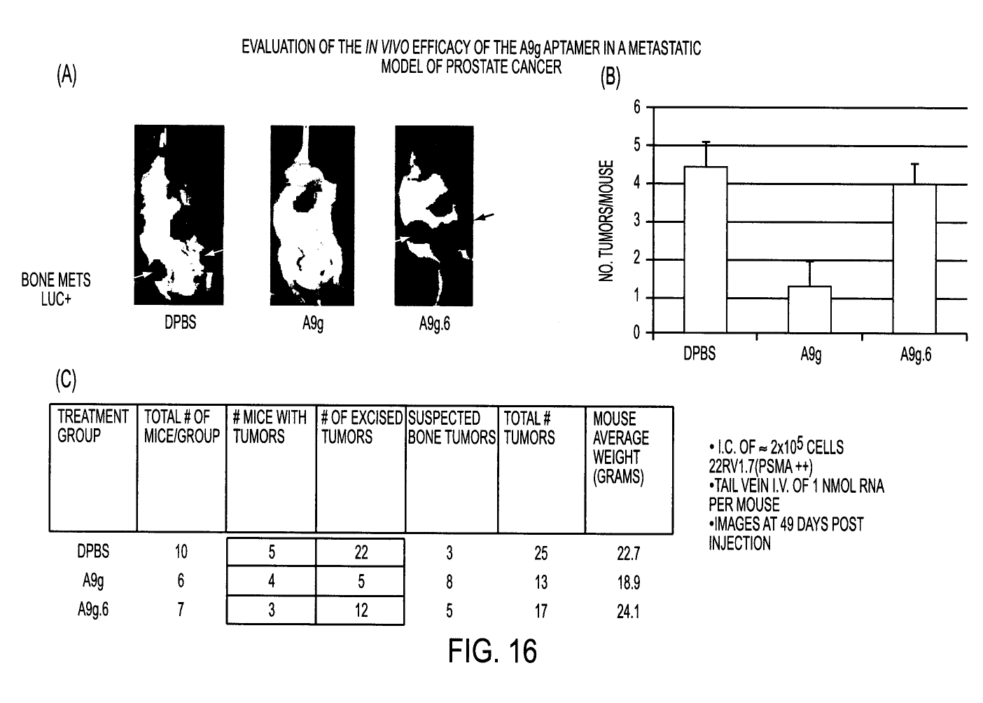

Experiments were performed to evaluate the effect of A9g on reducing motility and invasion of PSMA+ prostate cancer cells in culture and in vivo (FIGS. 16A-16C). These data demonstrate that aptamer A9g can inhibit metastases in a mouse model of prostate cancer. FIG. 16A shows representative images of mice treated with DPBS, A9g or A9g.6. The data from 16A are quantitated in 16B and the tumors excised from the mice reported in the table (FIG. 16C).

### Example 3

Biodistribution and Pharmacokinetic Data for A9g Aptamer

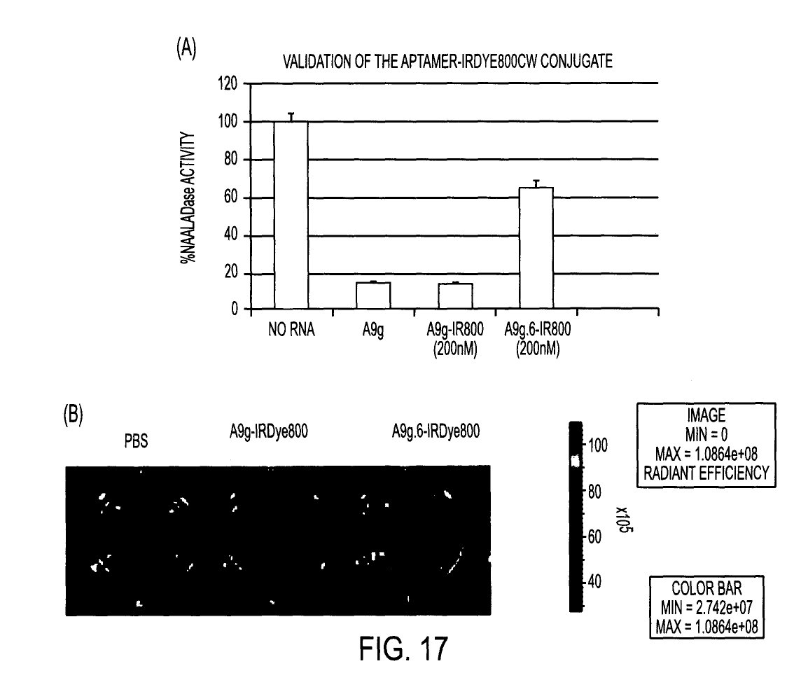

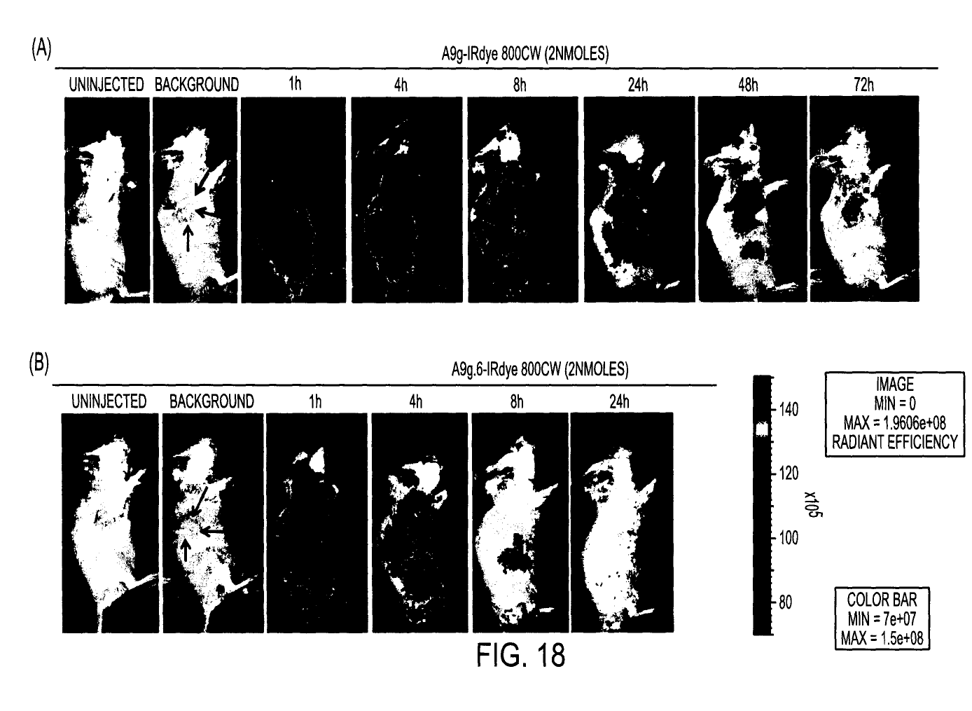

Experiments were performed to evaluate the biodistribution and pharmacokinetic data for the A9g aptamer (FIGS. 17A-17B and 18A-18B). FIG. 17A demonstrates that labeling the A9g aptamer with IR-Dye 800CW does not attenuate the NAALADase enzymatic activity of PSMA in vitro. Both A9g and A9g-IR800 inhibit the NAALADase reaction to a similar extent, as measured by the cleavage of ³H-labeled glutamate from [³H]NAAG by the PSMA enzyme. The labeled A9g.6 negative-control aptamer attenuates NAALADase activity to a much lesser degree than A9g. The fluorescence intensity of the labeled aptamers is shown in FIG. 17B (data obtained with Xenogen Ivis 200 system). The data is shown in FIG. 18A, which shows targeting of the infrared fluorophore-labeled A9g (A9g IR-Dye 800CW) in a PSMA-expressing prostate cancer xenograft mouse model (arrow). Images were acquired with an excitation filter of 710-760 nm and an emission filter of 810-875 nm on a Xenogen Ivis 200 system. Early time points show the distribution of the labeled aptamer throughout the body. By 72 hours, only the tumor xenograft shows significant uptake. FIG. 18B shows the biodistribution and pharmacokinetics of the non-PSMA targeting IR-Dye 800CW-labeled A9g.6 negative control aptamer. By 24 hours, the majority of the signal is cleared from the body and there is no significant uptake in the PSMA-expressing xenograft (arrow).

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nucleic acid aptamer molecule of 41 to 66 nucleotides in length comprising the nucleic acid sequence 5′-N₁GGRCCGAMAAAGVCCTGACTTCTATACTAAGBCTWCGYYCCN₂-3′ (SEQ ID NO:1),
where N₁ and N₂ can be present or absent, wherein when present N₁ is GGGAGGACGATGC and N₂ is AGACGACTCCC, N₁ is GGGACGATGC and N₂ is CAGACGCCC, N₁ is GGGATGC and N₂ is CAGACCC, N₁ is GGGACGATGC and N₂ is CAGACGACCC, N₁ is GGGC and N₂ is CACC, N₁ is GGGC and N₂ is CAGCCC, or N₁ is G and N₂ is C,
wherein R is a G or A nucleotide,
wherein M is a A or C nucleotide,
wherein V is a A, G or C nucleotide,
wherein B is a T, C or G nucleotide,
wherein W is a A or T nucleotide,
wherein Y is T or C nucleotide,
or its complement, or an RNA equivalent of the molecule or its complement.

2. A nucleic acid aptamer molecule consisting of A9a (SEQ ID NO:2), A9b (SEQ ID NO:3), A9c (SEQ ID NO:4), A9d (SEQ ID NO:5), A9e (SEQ ID NO:6), A9f (SEQ ID NO:7), A9g (SEQ ID NO:8), A9g.1 (SEQ ID NO:9), A9g.2 (SEQ ID NO:10), A9g.4 (SEQ ID NO:11), A9g.9/A9C (SEQ ID NO:12), or A9L (SEQ ID NO:13), or its complement, or an RNA equivalent of the molecule or its complement.

3. (canceled)

4. (canceled)

5. The nucleic acid aptamer molecule of claim 2 4, wherein the nucleic acid molecule is RNA.

6. The nucleic acid aptamer molecule of claim 2, wherein the nucleic acid molecule includes a modified nucleotide.

7. A conjugate comprising the nucleic acid aptamer molecule of claim 2 linked to a therapeutic or diagnostic molecule.

8. (canceled)

9. (canceled)

10. The conjugate of claim 7, wherein the nucleic acid aptamer molecule is linked to a therapeutic molecule, and the therapeutic molecule is a siRNA molecule.

11. The conjugate of claim 7, which further comprises a PEG molecule.

12. The conjugate of claim 11, wherein the PEG molecule has an average molecular weight of about 10 to 100 kDa in size.

13. A coding nucleic acid molecule encoding the nucleic acid aptamer molecule of claim 2.

14. An expression cassette comprising a promoter and the coding molecule of claim 13.

15. (canceled)

16. A viral vector comprising the expression cassette of claim 14.

17. An isolated or non-human cell comprising the PMSA receptor and a molecule of claim 2.

18. A method for delivering a therapeutic or diagnostic molecule to a cell having a PMSA receptor, comprising contacting the cell with the conjugate of claim 7.

19. A pharmaceutical composition comprising a molecule of claim 2 and a pharmaceutically acceptable carrier.

20. A method for treating a patient having cancer comprising administering a molecule of claim 2 to the patient.

21. A method for determining whether a patient has cancer comprising administering conjugate of claim 7 to the patient and determining whether the patient has cancer.

22. (canceled)

23. The method of claim 20, wherein the cancer is a solid sarcoma or carcinoma.

24. The method of claim 20, wherein the cancer is prostate cancer.

25. (canceled)

26. (canceled)

27. The aptamer of claim 2, wherein the aptamer is capable of binding to PSMA.

28. The aptamer of claim 2, wherein the aptamer is capable of inhibiting PSMA enzymatic activity.
